SEQUENCE LISTING (30 sequences)

SEQ ID NO: 1 - DNA, 10750 bp, Artificial Sequence
  Synthetic Construct
gtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggaggcggaggatctggcggcggaggaagtggcggcggaggatctggcggcggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcggaggcggaggatctggcggcggaggaagtggcggcggaggatctggcggcggaggaagcgccgtgcagctggtgcagagcggagccgaggtgaagaagcccggcagcagcgtcaaggtgtcctgcaaggccagcggctacagcttcaccggctacaacatgaactgggtgcggcaggccccaggccagggactggaatggatgggcaacatcgacccctactacggcggcaccacctacaaccggaagttcaagggccgggtgaccctgaccgtggacaagagcagcagcaccgcctacatggaactgagcagcctgcggagcgaggacaccgccgtgtactactgcgccagatccgtgggccccatggactactggggccagggcaccctggtcaccgtctcttcaggaggcggaggatctggcggcggaggaagtggcggcggaggatctggcggcggaggaagcgacatccagatgacccagagccccagcagcctgagcgccagcgtgggcgaccgggtgaccatcacctgccggaccagcgagaacgtgtacagctacctggcctggtatcagcagaagcccggcaaggcccccaagctgctggtgtcctccgccaagaccctggccgagggcgtgcccagccggtttagcggcagcggctccggcaccgacttcaccctgaccatcagcagcctgcagcccgaggacttcgccacctacttttgccagcaccacagcgacaacccctggaccttcggccagggcaccaaggtggagatcaaacgtaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtccggaggcggcggagagggcagaggaagtcttctaacatgcggtgacgtggaggagaatcccggccctaggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctctaccgatcccgcccctaactccgcccagttccgcccattctccgccccatggctgactaattttttttatttatgcagaggccgaggccgcctcggcctctgagctattccagaagtagtgaggaggcttttttggaggcactcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgg

SEQ ID NO: 2 - DNA, 10750 bp, Artificial Sequence
  Synthetic Construct
gtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgccgtgcagctggtgcagagcggagccgaggtgaagaagcccggcagcagcgtcaaggtgtcctgcaaggccagcggctacagcttcaccggctacaacatgaactgggtgcggcaggccccaggccagggactggaatggatgggcaacatcgacccctactacggcggcaccacctacaaccggaagttcaagggccgggtgaccctgaccgtggacaagagcagcagcaccgcctacatggaactgagcagcctgcggagcgaggacaccgccgtgtactactgcgccagatccgtgggccccatggactactggggccagggcaccctggtcaccgtctcttcaggaggcggaggatctggcggcggaggaagtggcggcggaggatctggcggcggaggaagcgacatccagatgacccagagccccagcagcctgagcgccagcgtgggcgaccgggtgaccatcacctgccggaccagcgagaacgtgtacagctacctggcctggtatcagcagaagcccggcaaggcccccaagctgctggtgtcctccgccaagaccctggccgagggcgtgcccagccggtttagcggcagcggctccggcaccgacttcaccctgaccatcagcagcctgcagcccgaggacttcgccacctacttttgccagcaccacagcgacaacccctggaccttcggccagggcaccaaggtggagatcaaacgtggaggcggaggatctggcggcggaggaagtggcggcggaggatctggcggcggaggaagcgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggaggcggaggatctggcggcggaggaagtggcggcggaggatctggcggcggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtccggaggcggcggagagggcagaggaagtcttctaacatgcggtgacgtggaggagaatcccggccctaggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctctaccgatcccgcccctaactccgcccagttccgcccattctccgccccatggctgactaattttttttatttatgcagaggccgaggccgcctcggcctctgagctattccagaagtagtgaggaggcttttttggaggcactcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgg

SEQ ID NO: 3 - DNA, 741 bp, Artificial Sequence
  Synthetic Construct
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggaggcggaggatctggcggcggaggaagtggcggcggaggatctggcggcggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 4 - DNA, 732 bp, Artificial Sequence
  Synthetic Construct
gccgtgcagctggtgcagagcggagccgaggtgaagaagcccggcagcagcgtcaaggtgtcctgcaaggccagcggctacagcttcaccggctacaacatgaactgggtgcggcaggccccaggccagggactggaatggatgggcaacatcgacccctactacggcggcaccacctacaaccggaagttcaagggccgggtgaccctgaccgtggacaagagcagcagcaccgcctacatggaactgagcagcctgcggagcgaggacaccgccgtgtactactgcgccagatccgtgggccccatggactactggggccagggcaccctggtcaccgtctcttcaggaggcggaggatctggcggcggaggaagtggcggcggaggatctggcggcggaggaagcgacatccagatgacccagagccccagcagcctgagcgccagcgtgggcgaccgggtgaccatcacctgccggaccagcgagaacgtgtacagctacctggcctggtatcagcagaagcccggcaaggcccccaagctgctggtgtcctccgccaagaccctggccgagggcgtgcccagccggtttagcggcagcggctccggcaccgacttcaccctgaccatcagcagcctgcagcccgaggacttcgccacctacttttgccagcaccacagcgacaacccctggaccttcggccagggcaccaaggtggagatcaaacgt

SEQ ID NO: 5 - DNA, 126 bp, Artificial Sequence
  Synthetic Construct
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 6 - DNA, 336 bp, Artificial Sequence
  Synthetic Construct
cgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 7 - protein, 116 aa, Artificial Sequence
  Synthetic Construct
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSS

SEQ ID NO: 8 - protein, 108 aa, Artificial Sequence
  Synthetic Construct
DIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKR

SEQ ID NO: 9 - protein, 20 aa, Artificial Sequence
  Synthetic Construct
GGGGSGGGGSGGGGSGGGGS

SEQ ID NO: 10 - protein, 244 aa, Artificial Sequence
  Synthetic Construct
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSGGGGSGGGGSGGGGSGGGGSDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKR

SEQ ID NO: 11 - protein, 244 aa, Artificial Sequence
  Synthetic Construct
DIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRGGGGSGGGGSGGGGSGGGGSAVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSS

SEQ ID NO: 12 - protein, 488 aa, Artificial Sequence
  Synthetic Construct
MALPVTALLLPLALLLHAARPAVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSGGGGSGGGGSGGGGSGGGGSDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 488 aa, Artificial Sequence
  Synthetic Construct
MALPVTALLLPLALLLHAARPDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRGGGGSGGGGSGGGGSGGGGSAVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 21 aa, Artificial Sequence
  Synthetic Construct
MALPVTALLLPLALLLHAARP

SEQ ID NO: 15 - protein, 69 aa, Artificial Sequence
  Synthetic Construct
TTTPAPRPPTPAPTIASQPLSLRPEACRPAAGGAVHTRGLDFACDIYIWAPLAGTCGVLLLSLVITLYC

SEQ ID NO: 16 - protein, 42 aa, Artificial Sequence
  Synthetic Construct
KRGRKKLLYIFKQPFMRPVQTTQEEDGCSCRFPEEEEGGCEL

SEQ ID NO: 17 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 120 aa, Artificial Sequence
  Synthetic Construct
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 19 - protein, 107 aa, Artificial Sequence
  Synthetic Construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 20 - protein, 247 aa, Artificial Sequence
  Synthetic Construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 21 - protein, 755 aa, Artificial Sequence
  Synthetic Construct
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSAVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSGGGGSGGGGSGGGGSGGGGSDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 755 aa, Artificial Sequence
  Synthetic Construct
MALPVTALLLPLALLLHAARPAVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSGGGGSGGGGSGGGGSGGGGSDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 467 aa, Artificial Sequence
  Synthetic Construct
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSGGGGSGGGGSGGGGSGGGGSDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 467 aa, Artificial Sequence
  Synthetic Construct
DIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRGGGGSGGGGSGGGGSGGGGSAVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 734 aa, Artificial Sequence
  Synthetic Construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSAVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSGGGGSGGGGSGGGGSGGGGSDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 734 aa, Artificial Sequence
  Synthetic Construct
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSGGGGSGGGGSGGGGSGGGGSDIQMTQSPSSLSASVGDRVTITCRTSENVYSYLAWYQQKPGKAPKLLVSSAKTLAEGVPSRFSGSGSGTDFTLTISSLQPEDFATYFCQHHSDNPWTFGQGTKVEIKRGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 9967 bp, Artificial Sequence
  Synthetic Construct
gtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggaggcggaggatctggcggcggaggaagtggcggcggaggatctggcggcggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcggaggcggaggatctggcggcggaggaagtggcggcggaggatctggcggcggaggaagcgccgtgcagctggtgcagagcggagccgaggtgaagaagcccggcagcagcgtcaaggtgtcctgcaaggccagcggctacagcttcaccggctacaacatgaactgggtgcggcaggccccaggccagggactggaatggatgggcaacatcgacccctactacggcggcaccacctacaaccggaagttcaagggccgggtgaccctgaccgtggacaagagcagcagcaccgcctacatggaactgagcagcctgcggagcgaggacaccgccgtgtactactgcgccagatccgtgggccccatggactactggggccagggcaccctggtcaccgtctcttcaggaggcggaggatctggcggcggaggaagtggcggcggaggatctggcggcggaggaagcgacatccagatgacccagagccccagcagcctgagcgccagcgtgggcgaccgggtgaccatcacctgccggaccagcgagaacgtgtacagctacctggcctggtatcagcagaagcccggcaaggcccccaagctgctggtgtcctccgccaagaccctggccgagggcgtgcccagccggtttagcggcagcggctccggcaccgacttcaccctgaccatcagcagcctgcagcccgaggacttcgccacctacttttgccagcaccacagcgacaacccctggaccttcggccagggcaccaaggtggagatcaaacgtaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctctaccgatcccgcccctaactccgcccagttccgcccattctccgccccatggctgactaattttttttatttatgcagaggccgaggccgcctcggcctctgagctattccagaagtagtgaggaggcttttttggaggcactcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgg

SEQ ID NO: 28 - DNA, 9967 bp, Artificial Sequence
  Synthetic Construct
gtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgccgtgcagctggtgcagagcggagccgaggtgaagaagcccggcagcagcgtcaaggtgtcctgcaaggccagcggctacagcttcaccggctacaacatgaactgggtgcggcaggccccaggccagggactggaatggatgggcaacatcgacccctactacggcggcaccacctacaaccggaagttcaagggccgggtgaccctgaccgtggacaagagcagcagcaccgcctacatggaactgagcagcctgcggagcgaggacaccgccgtgtactactgcgccagatccgtgggccccatggactactggggccagggcaccctggtcaccgtctcttcaggaggcggaggatctggcggcggaggaagtggcggcggaggatctggcggcggaggaagcgacatccagatgacccagagccccagcagcctgagcgccagcgtgggcgaccgggtgaccatcacctgccggaccagcgagaacgtgtacagctacctggcctggtatcagcagaagcccggcaaggcccccaagctgctggtgtcctccgccaagaccctggccgagggcgtgcccagccggtttagcggcagcggctccggcaccgacttcaccctgaccatcagcagcctgcagcccgaggacttcgccacctacttttgccagcaccacagcgacaacccctggaccttcggccagggcaccaaggtggagatcaaacgtggaggcggaggatctggcggcggaggaagtggcggcggaggatctggcggcggaggaagcgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggaggcggaggatctggcggcggaggaagtggcggcggaggatctggcggcggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctctaccgatcccgcccctaactccgcccagttccgcccattctccgccccatggctgactaattttttttatttatgcagaggccgaggccgcctcggcctctgagctattccagaagtagtgaggaggcttttttggaggcactcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgg

SEQ ID NO: 29 - DNA, 479 bp, Artificial Sequence
  Synthetic Construct
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggaggcggaggatctggcggcggaggaagtggcggcggaggatctggcggcggaggaagccaggtccaactccaagaaagcggaccgggtcttgt

SEQ ID NO: 30 - DNA, 2265 bp, Artificial Sequence
  Synthetic Construct
atggccctccctgtcaccgccctgctgcttccgctggctcttctgctccacgccgctcggcccgccgtgcagctggtgcagagcggagccgaggtgaagaagcccggcagcagcgtcaaggtgtcctgcaaggccagcggctacagcttcaccggctacaacatgaactgggtgcggcaggccccaggccagggactggaatggatgggcaacatcgacccctactacggcggcaccacctacaaccggaagttcaagggccgggtgaccctgaccgtggacaagagcagcagcaccgcctacatggaactgagcagcctgcggagcgaggacaccgccgtgtactactgcgccagatccgtgggccccatggactactggggccagggcaccctggtcaccgtctcttcaggaggcggaggatctggcggcggaggaagtggcggcggaggatctggcggcggaggaagcgacatccagatgacccagagccccagcagcctgagcgccagcgtgggcgaccgggtgaccatcacctgccggaccagcgagaacgtgtacagctacctggcctggtatcagcagaagcccggcaaggcccccaagctgctggtgtcctccgccaagaccctggccgagggcgtgcccagccggtttagcggcagcggctccggcaccgacttcaccctgaccatcagcagcctgcagcccgaggacttcgccacctacttttgccagcaccacagcgacaacccctggaccttcggccagggcaccaaggtggagatcaaacgtggaggcggaggatctggcggcggaggaagtggcggcggaggatctggcggcggaggaagcgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggaggcggaggatctggcggcggaggaagtggcggcggaggatctggcggcggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

(19) United States
(12) Patent Application Publication
(10) Pub. No.: US 2021/0268027 A1
(43) Pub. Date: Sep. 2, 2021

(54) CHIMERIC ANTIGEN RECEPTORS TARGETING CD37 AND CD19

(71) Applicant: The General Hospital Corporation, Boston, MA (US)

(72) Inventor: Marcela V. Maus, Lexington, MA (US)

(73) Assignee: The General Hospital Corporation, Boston, MA (US)

(21) Appl. No.: 17/254,392

(22) PCT Filed: Jun. 21, 2019
(86) PCT No.: PCT/US19/38518

§ 371 Date: Dec. 21, 2020

Related U.S. Application Data
(60) Provisional application No. 62/688,775, filed on Jun. 22, 2018.
(60) Provisional application No. 62/757,562, filed on Nov. 8, 2018.

(51) Int. Cl. A61K 35/17; C07K 16/28; C07K 14/705; C07K 14/725; C12N 5/0783; A61P 35/00

(52) U.S. Cl. CPC A61K 35/17; C12N 2510/00; C07K 16/2803; C07K 14/70517; C07K 14/7051; C07K 14/70578; C12N 5/0636; C12N 5/0646; A61P 35/00; C07K 2319/03; C07K 2319/02; C07K 2319/33; C07K 2317/31; C07K 2317/622; C07K 2317/53; C07K 2317/56; C07K 16/2896

(57) ABSTRACT

The invention provides bispecific chimeric antigen receptors (CARs) targeting CD37 and CD19, as well as related molecules, immune cells including the same, compositions thereof, and their methods of use. The invention further provides methods for treating a disease or disorder, e.g., a cancer.

### CROSS-REFERENCE TO RELATED APPLICATIONS

This application claims benefit of U.S. Provisional Application No. 62/688,775, filed Jun. 22, 2018, and U.S. Provisional Application No. 62/757,562, filed Nov. 8, 2018, the contents of which are incorporated herein by reference in their entirety.

### SEQUENCE LISTING

The instant application contains a Sequence Listing which has been submitted electronically in ASCII format and is hereby incorporated by reference in its entirety. The ASCII copy, created on Jun. 19, 2019, is named 51295-018WO3_Sequence_Listing_6.19.19_ST25 and is 56,545 bytes in size.

### BACKGROUND OF THE INVENTION

Immunotherapy employs the use of a patient's immune system to treat disease, such as a cancer, an autoimmune disease, or a plasma cell disorder. Adoptive cell transfer employs the use of antigen-specific immune cells, such as T cells, to treat such diseases. The immune cells used in this therapy can be modified to exhibit a desired specificity, e.g., by expressing a chimeric antigen receptor (CAR). CARs provide a way to direct a cytotoxic T cell or NK cell response to target cells expressing a selected target antigen, most often a tumor antigen or tumor-associated antigen, and are an adaptation of the T cell receptor, where the antigen binding domain is replaced with the antigen binding domain of an antibody specific to the target antigen. Engagement of the target antigen on the surface of a target cell by a CAR expressed on a T cell promotes killing of the target cell. The use of CAR-expressing T cells for treatment of disease is known as CAR T cell immunotherapy.

Thus far, two CAR T cell products have been approved for the treatment of relapsed or refractory large cell lymphomas, both of which target CD19: axicabtagene ciloleucel, which bears the CD28 costimulatory domain, and tisagenlecleucel, which bears the 4-1 BB costimulatory domain. Tisagenlecleucel has also been approved for the treatment of relapsed or refractory acute B cell lymphoblastic leukemia (ALL) in children and young adults. Anti-CD19 CAR T cell treatment has effected responses in the 60-80% range, and approximately 40% of patients have achieved long-term complete remissions. However, disease relapse due to CD19 antigen target loss has been observed in all patient subsets, including both acute lymphoblastic (ALL) and non-Hogdkin lymphoma (NHL) patients (Maude et al., N. Engl. J. Med. 371:1507-17, 2014; Evans et al., Br. J. Haematol. 171(2):205-209, 2015; Schuster et al., N. Engl. J. Med. 377(26):2545-2554, 2017). There exists an unmet clinical need for improved treatments.

Furthermore, patients with T cell lymphomas are not candidates for anti-CD19 CAR T therapy. For example, peripheral T-cell lymphomas (PTCLs) are an aggressive heterogeneous group of tumors that represent 12-15% of all non-Hodgkin lymphomas. Despite the recognition of their complex heterogeneity and the discovery of recurrent defects, PTCLs remain a clinical dilemma and poorly treated. While CAR T immunotherapy has demonstrated impressive clinical results in ALL and B cell NHL, there has yet to be success demonstrated in treating T cell malignancies.

Thus, there exists a need for new or improved treatments for B and T cell malignancies.

### SUMMARY OF THE INVENTION

The invention provides, inter alia, bispecific chimeric antigen receptors (CARs) targeting CD37 and CD19 for the use in treating a disease or disorder described herein, e.g., cancer.

In one aspect, the invention features a chimeric antigen receptor (CAR) including (i) an extracellular domain including a CD37-binding domain and a CD19-binding domain, (ii) a transmembrane domain, and (iii) an intracellular signaling domain.

In some embodiments, the CD37-binding domain and/or the CD19-binding domain includes an antibody, or an antigen binding fragment thereof, e.g., a single chain variable fragment (scFv). In some embodiments, the CD19-binding domain is positioned N-terminal to the CD37-binding domain, or the CD37-binding domain is positioned N-terminal to the CD19-binding domain.

In some embodiments, the CAR further includes (iv) one or more co-stimulatory domains. In some embodiments, the transmembrane domain includes a hinge/transmembrane domain, e.g., of CD8 or 4-1 BB. In particular embodiments, the hinge/transmembrane domain includes the hinge/transmembrane domain of CD8, optionally including the amino acid sequence of SEQ ID NO: 9.

In further embodiments, the intracellular signaling domain includes the intracellular signaling domain of TCRζ, FcRγ, FcRβ, CD3γ, CD3θ, CD3ε, CD3ζ, CD22, CD79a, CD79b, or CD66d. In particular embodiments, the intracellular signaling domain includes the intracellular signaling domain of CD3ζ, optionally including the amino acid sequence of SEQ ID NO: 11. In some embodiments, the co-stimulatory domain includes the co-stimulatory domain of 4-1 BB, CD28, or OX-40. In particular embodiments, the co-stimulatory domain includes the co-stimulatory domain of 4-1 BB, optionally including the amino acid sequence of SEQ ID NO: 10.

In some embodiments, the CAR includes an amino acid sequence having at least 90% (e.g., 91%, 92%, 93%, 94%, 95%, 96%, 97%, 98%, or 99%) sequence identity to, or including the sequence of, the amino acid sequence of SEQ ID NO: 15, 16, 19, or 20.

In some embodiments, the CD37-binding domain includes a heavy chain variable domain (VH) including an amino acid sequence having at least 90% (e.g., 91%, 92%, 93%, 94%, 95%, 96%, 97%, 98%, or 99%) sequence identity to, or including the sequence of, the amino acid sequence of SEQ ID NO: 1 and a light chain variable domain (VL) including an amino acid sequence having at least 90% (e.g., 91%, 92%, 93%, 94%, 95%, 96%, 97%, 98%, or 99%) sequence identity to, or including the sequence of, the amino acid sequence of SEQ ID NO: 2. In some embodiments, the VH is positioned N-terminal to the VL, or the VL is positioned N-terminal to the VH. In further embodiments, the CD37-binding domain includes an amino acid sequence having at least 90% (e.g., 91%, 92%, 93%, 94%, 95%, 96%, 97%, 98%, or 99%) sequence identity to, or including the sequence of, the amino acid sequence of SEQ ID NO: 4 or 5.

In further embodiments, the CD19-binding domain includes a heavy chain variable domain (VH) including an amino acid sequence having at least 90% (e.g., 91%, 92%, 93%, 94%, 95%, 96%, 97%, 98%, or 99%) sequence identity to, or including the sequence of, the amino acid sequence of SEQ ID NO: 12 and a light chain variable domain (VL) including an amino acid sequence having at least 90% (e.g., 91%, 92%, 93%, 94%, 95%, 96%, 97%, 98%, or 99%) sequence identity to, or including the sequence of, the amino acid sequence of SEQ ID NO: 13. In some embodiments, the CD19-binding domain includes an amino acid sequence having at least 90% (e.g., 91%, 92%, 93%, 94%, 95%, 96%, 97%, 98%, or 99%) sequence identity to, or including the sequence of, the amino acid sequence of SEQ ID NO: 14.

In another aspect, the invention features a polynucleotide encoding the CAR of any of the preceding aspects.

In some embodiments, the polynucleotide further includes a suicide gene. In some embodiments, the polynucleotide further includes a sequence encoding a signal sequence.

In another aspect, the invention features an immune cell including the CAR and/or the polynucleotide of any of the preceding aspects.

In some embodiments, the immune cell (e.g., a human cell) is a T cell or a natural killer (NK) cell.

In another aspect, the invention features a pharmaceutical composition including the immune cell of any of the preceding aspects and a pharmaceutically acceptable carrier.

In another aspect, the invention features a method of treating a cancer in a subject in need thereof, the method including administering the immune cell of any one of the preceding aspects, or a pharmaceutical composition thereof, to the subject.

In some embodiments, the cancer expresses CD37. In some embodiments, the cancer is a B cell non-Hodgkin lymphoma (e.g., mantle cell lymphoma (MCL), diffuse large B cell lymphoma (DLBCL), follicular lymphoma (FL), or Burkitt's lymphoma), a T cell lymphoma (e.g., peripheral T cell lymphoma (PTCL), cutaneous T cell lymphoma (CTCL), angioimmunoblastic T cell lymphoma (AITL), or anaplastic large cell lymphoma (ALCL)), or a leukemia (e.g., chronic lymphocytic leukemia (CLL)).

In some embodiments, the subject is non-responsive to anti-CD19 therapy. In further embodiments, the subject is co-administered anti-CD19 therapy.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of each of which are all incorporated by reference herein in their entireties.

The terms “decrease,” “reduced,” “reduction,” or “inhibit” are all used herein to mean a decrease by a statistically significant amount. In some embodiments, “reduce,” “reduction,” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

The terms “increased,” “increase,” “enhance,” or “activate” are all used herein to mean an increase by a statically significant amount. In some embodiments, the terms “increased,” “increase,” “enhance,” or “activate” can mean an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease, e.g., cancer. A subject can be male or female.

A subject can be one who has been previously diagnosed with or identified as suffering from or having a condition in need of treatment (e.g., a lymphoma, leukemia or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

A “disease” is a state of health of an animal, for example, a human, wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “tumor antigen” and “cancer antigen” are used interchangeably to refer to antigens that are differentially expressed by cancer cells and can thereby be exploited in order to target cancer cells. Cancer antigens are antigens that can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively.

As used herein, the term “chimeric” refers to the product of the fusion of portions of at least two or more different polynucleotide molecules. In one embodiment, the term “chimeric” refers to a gene expression element produced through the manipulation of known elements or other polynucleotide molecules.

In some embodiments, “activation” can refer to the state of an immune cell, e.g., a T or NK cell that has been sufficiently stimulated to induce detectable cellular proliferation. In some embodiments activation can refer to induced cytokine production. In other embodiments, activation can refer to detectable effector functions. At a minimum, an activated T or NK cell as used herein is a proliferative T or NK cell.

As used herein, the terms “specific binding” and “specifically binds” refer to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

A “stimulatory ligand,” as used herein, refers to a ligand that when present on an antigen presenting cell (APC, e.g., a macrophage, a dendritic cell, a B-cell, an artificial APC, and the like) can specifically bind with a cognate binding partner (referred to herein as a “stimulatory molecule” or “co-stimulatory molecule”) on a T cell, thereby mediating a primary response by the T cell, including, but not limited to, proliferation, activation, initiation of an immune response, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

A “stimulatory molecule,” as the term is used herein, means a molecule on a T cell that specifically binds with a cognate stimulatory ligand present on an antigen presenting cell.

“Co-stimulatory ligand,” as the term is used herein, includes a molecule on an APC that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, 4-1 BBL, OX40L, CD7, B7-1 (CD80), B7-2 (CD86), PD-L1, PD-L2, inducible COStimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll-like receptor and a ligand that specifically binds with B7-H3. A co-stimulatory ligand also can include, but is not limited to, an antibody that specifically binds with a co-stimulatory molecule present on a T cell, such as, but not limited to, CD27, CD28, 4-1 BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA, a Toll-like receptor, CD27, CD28, 4-1 BB, OX40, CD30, CD40, PD-1, ICOS, lymphocyte function-associated antigen-1 (LFA-1), CD2, CD7, LIGHT, NKG2C, B7-H3, and CD83.

In one embodiment, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene. The term “cell” or “engineered cell” and their grammatical equivalents as used herein can refer to a cell of human or non-human animal origin.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide that retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions, or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, a nucleic acid encoding a polypeptide as described herein (e.g., a bispecific CAR polypeptide) is comprised by a vector. In some of the aspects described herein, a nucleic acid sequence encoding a given polypeptide as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example, in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, a “signal peptide” or “signal sequence” refers to a peptide at the N-terminus of a newly synthesized protein that serves to direct a nascent protein into the endoplasmic reticulum. In some embodiments, the signal peptide is a CD8 signal peptide.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

By “recombinant vector” is meant a vector that includes a heterologous nucleic acid sequence or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra-chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down, or stop the progression or severity of a condition associated with a disease or disorder, e.g. acute lymphoblastic leukemia or other cancer, disease, or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

As used herein, the term “administering,” refers to the placement of a therapeutic or pharmaceutical composition as disclosed herein into a subject by a method or route that results in at least partial delivery of the agent at a desired site. Pharmaceutical compositions comprising agents as disclosed herein can be administered by any appropriate route that results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined within the description of the various aspects and embodiments of the technology of the following.

### BRIEF DESCRIPTION OF THE DRAWINGS

FIG. 1A shows FACS plots of tumor cell lines stained with CD37 and CD19 antibodies. NALM6: acute lympoblastic leukemia (ALL) cell line; K562 expressing CD19 and CD37: positive control; JEKO-1: mantle cell lymphoma (MCL) cell line; RAJI: Burkitt's lymphoma cell line.

FIG. 1B shows FACS plots of samples derived from three MCL patients.

FIG. 1C shows the mean fluorescence intensity (MFI) of CD19 and CD37 on an MCL patient's cells. Each dot represents a separate xenograft sample. (n=3; medians shown).

FIG. 1D shows CD19 and CD37 expression on peripheral blood mononuclear cells (PBMCs) from CLL patients gated on CD3-lymphocytes (n=20; mean±S.D. shown; **** indicates p<0.0001 by t test).

FIG. 1E shows CD19 and CD37 on CLL samples. Expression level of CD19 and CD37 on 21 patients with CLL by flow cytometry gated on the CD3-CD20+ B cells. Mean±S.D. shown.

FIG. 2A shows distribution of CD19 and CD37 antigens on CLL PBMCs. Antibodies bound per cell (ABC) gated on CD3− cells from each patient samples is shown.

FIG. 2B shows CD19 and CD37 antigen density. CD19 range: 24396-70952, mean: 43238; CD37 range: 8992-46550, mean: 23989

FIG. 2C shows CD37 immunohistochemistry in primary ALK-negative (left) and ALK-positive (right) ALCL specimens from the tissue microarray.

FIG. 3A shows a schematic diagram of two anti-CD37 second generation CAR constructs with different orientations of a humanized murine antibody-derived single-chain variable fragment: the light-to-heavy orientation (CAR-37 L-H, top) and the heavy-to-light (CAR-37 H-L, bottom).

FIG. 3B shows representative flow plots of primary human T cells transduction efficiency after 10 days of activation with CD3/CD28 beads.

FIG. 3C shows expanded T cells from three healthy donors included variable CAR-37 expression with a mean of 38% (L-H) and 75% (H-L).

FIG. 3D shows ex vivo expansion of CD3/CD28 bead-activated and target-stimulated T cells using static culture conditions in three healthy donors for 38 days. Each arrow represents antigen stimulation with K562 cells transduced to express CD37 and CD19.

FIG. 3E shows activation of Jurkat reporter (NFAT-Luc) T cells transduced with different CAR constructs and co-cultured with tumor cells. Luciferase activity was measured after 16 hours. (CD3-CD28 beads: positive control).

FIGS. 3F-3I show whole blood from six normal donors stained with CD45, CD3ζ, CD19, CD16, CD14, CD56, and CD37 antibodies or isotype control. FIG. 3F shows histograms representing CD37 expression gated on CD45+CD19+ B cells. FIG. 3G shows histograms representing CD37 expression on gated CD45+CD16+CD14+ monocyte population. FIG. 3H shows histograms representing CD37 expression on gated T cells (CD45+CD3+). FIG. 3I shows histograms representing CD37 expression on gated CD45+CD16+CD56+NK cells.

FIG. 4A shows the number of CSFE labeled, unstimulated target T cells measured by flow cytometry after 24 hours of co-culture at indicated E:T ratio with CAR-37 H-L, CAR-19, or untransduced T cells.

FIG. 4B shows the number of CSFE labeled T cells stimulated with PMA/ionomycin for 6 hours, then measured by flow cytometry after 24 hours of co-culture at indicated E:T ratio with CAR-37 H-L, CAR-19, or untransduced T cells.

FIG. 4C shows the number of Jeko-1 CBG-GFP cells measured by flow cytometry after 24 hours of co-culture at indicated E:T ratio with CAR-37 H-L, CAR-19, or untransduced T cells. Bars indicate mean±S.E.M. count of triplicates from one normal donor, representative of 3 normal donors.

FIG. 4D shows CD107a and IFNγ production relative to media by CAR-37 H-L and CAR-19 T cells incubated with primary immune cells for 6 hours at 1:1 E:T ratio was analyzed by flow cytometry. Bars show mean±S.E.M. percentage of the three normal donors analyzed.

FIG. 5 shows the cytotoxic capacity of CAR-37 T cells measured after overnight co-culture with targets. CAR T cells were co-cultured at indicated E:T ratios with indicated tumor cell lines. Increasing concentration of CAR-37 and CAR-19 T cells led to specific killing, while no killing was observed in the control group (UTD). The cytotoxicity assay is representative of three independent experiments conducted with different healthy donors.

FIGS. 6A and 6B show cytokine production by CAR-37 H-L, CAR-19, or UTD T cells incubated with primary CLL (FIG. 6A) or MCL PDX (FIG. 6B) tumor samples. CAR T cells were incubated with target cells for 24 hours at a 1:1 E:T ratio, and culture supernatants were analyzed by Luminex assay. Data are plotted as mean±S.E.M. for three donors.

FIG. 6C shows cytokine production by CAR-37, CAR-19, or UTD T cells incubated with indicated tumor cell lines for 24 hours at 1:1 E:T ratio; culture supernatants were analyzed by Luminex assay. Significant production of several cytokines is noted in CAR-37 and CAR-19 groups, but not in UTD. Three normal donors analyzed, mean±S.E.M. is shown.

FIG. 6D shows IL-6 production by CAR-37, CAR-19, or UTD T cells incubated with indicated tumor cell lines for 24 hours at 1:1 E:T ratio; culture supernatants were analyzed by Luminex assay. Three normal donors analyzed, mean±S.E.M. is shown.

FIGS. 7A-7C show direct comparison of anti MCL activity of CAR-37 T cells in MCL tumor model. FIG. 7A shows an experimental schematic: NSG mice were injected IV with 1×10⁶ JEKO-1 (CBG-GFP) cells and monitored by BLI for tumor burden at different time points. At day 0, mice were randomized based on tumor burden (BLI) to receive 1×10⁶ control T cells (UTD), CAR-37 L-H, or CAR-37 H-L. FIG. 7B shows representative bioluminescent images of JEKO-1 growth over time. FIG. 7C shows average radiance (p/s/cm²/sr) of whole mice in the three groups at different time points. Graph is representative of one experiment with five mice per group. Mean±S.D. is shown.

FIG. 7D shows a schematic diagram of the experimental design. NSG mice were injected IV with 1×10⁶ JEKO-1 (CBG-GFP) cells and monitored by BLI for tumor burden at different time points. At day 0, mice were randomized based on tumor burden (BLI) to receive 2×10⁶ control T cells (UTD), CAR-37 or CAR-19.

FIG. 7E shows representative bioluminescent images of JEKO-1 growth over time.

FIG. 7F shows average flux (photons/second) of whole mice in the three groups at different time points. Graph is representative of two experiments with five mice per group, conducted with CAR T cells obtained from two different healthy donors. Mean±S.D. shown, *** indicates p<0.001 by two-way Anova test.

FIG. 7G shows the absolute numbers of CAR T cells, which were monitored by bleeding and flow cytometric detection. Absolute CAR T cell counts in peripheral blood at day 14 after CART injection are shown. * indicates p<0.05 by t test.

FIG. 8A shows a schematic diagram of the experimental design. NSG mice were injected IV with 1×10⁶ MCL patient-derived cells and monitored for tumor burden by bioluminescent imaging (BLI) over time. At day 0, mice were randomized based on tumor burden to receive 3×10⁶ control T cells (UTD), CAR-37 or CAR-19.

FIG. 8B shows representative BLI of MCL xenografts over time.

FIG. 8C shows the average flux (photons/second) of whole mice in the three groups at different time points. Graph is representative two simultaneous experiments of five mice per group, conducted with CAR T cells obtained from two different healthy donors, and pooled data. Mean±S.D. shown (t test, p<0.05).

FIG. 8D shows the absolute numbers of CAR T cells were monitored in peripheral blood using flow cytometry. Absolute counts of CART cells are plotted at day 14.

FIG. 9A shows CD37 expression on PTCL tumor cell lines.

FIG. 9B shows representative FACS plots from patient derived samples.

FIGS. 10A and 10B show CD69 expression (FIG. 10A) and CD107a degranulation (FIG. 10B) of CAR T cells as evaluated by flow cytometry after 6 hours of co-culture with indicated tumor cells at 1:1 E:T ratio. Degranulation is relative to PMA positive control; representative normal donor is shown.

FIGS. 10C and 10D show the cytotoxic capacity of CAR-37 T cells, which was measured after overnight co-culture with Hut78 (FIG. 10C) and FEPD (FIG. 10D) target cells at different E:T ratio. The cytotoxicity assay is representative of three independent experiments conducted with different healthy donors. Mean±S.E.M. shown.

FIG. 11A shows a schematic diagram of two bispecific second generation CAR constructs with different order of the scFvs. CAR-19-37 (top) and CAR-37-19 (bottom).

FIG. 11B shows representative flow plots of primary human T cells transduction efficiency after 10 days of activation with CD3/CD28 beads.

FIG. 11C shows expanded T cells from two healthy donors, which showed variable CAR expression with a mean of 19% (CAR-19-37) and 48% (CAR-37-19).

FIG. 12A shows the activation of Jurkat reporter (NFAT-Luc) T cells transduced with different CAR constructs and co-cultured with tumor cells. Luciferase activity was measure after 16 hours. (CD3-CD28 beads: positive control).

FIG. 12B shows ex vivo expansion of CD3/CD28 bead-activated and target-stimulated T cells in two healthy donors for 30 days.

FIG. 12C shows the cytotoxic capacity of bispecific CAR T cells, which was measured after overnight co-culture with K562 targets transduced with CD37, CD19, or both, at indicated E:T ratios. The cytotoxicity assay is representative of two independent experiments conducted with different healthy donors.

FIG. 12D shows tumor burden in NSG mice over time. NSG mice were injected IV with 1×10⁶ JEKO-1(CBG-GFP) cells and monitored by BLI for tumor burden. At day 0, mice were randomized based on tumor burden (BLI) to receive 2×10⁶ control T cells (UTD), CAR-37, CAR-19, CAR-19-37 or CAR-37-19. All CAR T cell groups were normalized to have the same % CAR+ cells and untransduced cells. Average flux (photons/second) of whole mice at different time points is shown. Graph shows one experiment with six mice per group.

FIG. 12E shows absolute counts of CAR T cells, which were enumerated in peripheral blood by flow cytometry at the indicated time points. Absolute counts of CAR T cells are shown as mean±S.E.M.

### DETAILED DESCRIPTION OF THE INVENTION

The invention provides bispecific chimeric antigen receptor (CAR) polypeptides targeting CD37 and CD19. Also described are the nucleic acid molecules encoding the bispecific CARs, vectors including said nucleic acid molecules, and methods of making and using the same. Furthermore, provided are methods of treating a disease or disorder described herein, e.g., cancer, with the bispecific CARs and related molecules described herein.

### Chimeric Antigen Receptors

The technology described herein provides bispecific CARs targeting CD37 and CD19 for use in immunotherapy, e.g., for treating cancer.

The term “chimeric antigen receptor” or “CAR” as used herein refers to an engineered T cell receptor, which graft a ligand or antigen specificity onto T cells (e.g., naïve T cells, central memory T cells, effector memory T cells or combinations thereof) or NK cells. CARs are also known as artificial T-cell receptors, chimeric T cell receptors, or chimeric immunoreceptors. Furthermore, the term “CAR” includes the bispecific CARs as described herein.

A CAR places a chimeric extracellular target-binding domain that specifically binds a target, e.g., a polypeptide, expressed on the surface of a cell to be targeted for a T cell response onto a construct including a transmembrane domain and intracellular domain(s) of a T cell receptor molecule. In one embodiment, the chimeric extracellular target-binding domain comprises the antigen-binding domain(s) of an antibody that specifically binds an antigen expressed on a cell to be targeted for a T cell response. The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide CD3zeta (CD3) signals upon antigen binding. So-called “second-generation” CARs include those that provide both co-stimulation (e.g., CD28 or CD137) and activation (CD3) domains, and so-called “third-generation” CARs include those that provide multiple costimulatory (e.g., CD28 and CD137) domains and activation domains (e.g., CD3). In various embodiments, the CAR is selected to have high affinity or avidity for the target/antigen—for example, antibody-derived target or antigen binding domains will generally have higher affinity and/or avidity for the target antigen than would a naturally-occurring T cell receptor. This property, combined with the high specificity one can select for an antibody provides highly specific T cell targeting by CAR T cells.

As used herein, a “CAR T cell” or “CAR-T” refers to a T cell that expresses a CAR. Likewise, a “CAR NK cell” refers to an NK cell expressing a CAR. When expressed in a T or NK cell, CARs have the ability to redirect T or NK cell specificity and reactivity toward a selected target in a non-MHC-restricted manner, exploiting the antigen-binding properties of monoclonal antibodies. The non-MHC-restricted antigen recognition gives T or NK cells expressing CARs the ability to recognize an antigen independent of antigen processing, thus bypassing a major mechanism of tumor escape.

As used herein, the term “extracellular target binding domain” refers to a polypeptide found on the outside of the cell that is sufficient to facilitate binding to a target. The extracellular target binding domain will specifically bind to its binding partner, i.e., the target. As non-limiting examples, the extracellular target-binding domain can include an antigen-binding domain of an antibody, or a ligand, which recognizes and binds with a cognate binding partner (e.g., CD37 or CD19) protein. In this context, a ligand is a molecule that binds specifically to a portion of a protein and/or receptor. The cognate binding partner of a ligand useful in the methods and compositions described herein can generally be found on the surface of a cell. Ligand:cognate partner binding can result in the alteration of the ligand-bearing receptor, or activate a physiological response, for example, the activation of a signaling pathway. In one embodiment, the ligand can be non-native to the genome. Optionally, the ligand has a conserved function across at least two species. In one embodiment, the extracellular target binding domain comprises a non-antibody ligand.

Antibody Reagents

In various embodiments, the CARs described herein comprise an antibody reagent or an antigen-binding domain thereof as an extracellular target binding domain.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g., de Wildt et al., Eur J. Immunol. 26(3):629-639, 1996; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like. Fully human antibody binding domains can be selected, for example, from phage display libraries using methods known to those of ordinary skill in the art.

The V_{H} and V_{L} regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A. et al. (1991) Sequences of Proteins of Immunological Interest, Fifth Edition, U.S. Department of Health and Human Services, NIH Publication No. 91-3242, and Chothia et al., J. Mol. Biol. 196:901-917, 1987; each of which are incorporated by reference herein in their entireties). Each VH and V_{L} is typically composed of three CDRs and four FRs, arranged from amino-terminus to carboxy-terminus in the following order: FR1, CDR1, FR2, CDR2, FR3, CDR3, FR4.

In some embodiments, the antibody or antibody reagent is not a human antibody or antibody reagent, (i.e., the antibody or antibody reagent is mouse), but has been humanized. A “humanized antibody or antibody reagent” refers to a non-human antibody or antibody reagent that has been modified at the protein sequence level to increase its similarity to antibody or antibody reagent variants produced naturally in humans. One approach to humanizing antibodies employs the grafting of murine or other non-human CDRs onto human antibody frameworks.

In some embodiments, the extracellular target binding domain of a CAR comprises or consists essentially of a single-chain variable fragment (scFv) created by fusing the V_{H} and V_{L} domains of an antibody, generally a monoclonal antibody, via a flexible linker peptide. In various embodiments, the scFv is fused to a transmembrane domain, optionally via a hinge, and to a T cell receptor intracellular signaling domain, e.g., an engineered intracellular signaling domain as described herein. Antibody binding domains useful for the CARs described herein and ways to select and clone them are well-known to those of ordinary skill in the art.

In some embodiments, the CARs useful in the technology described herein comprise at least two antigen-specific targeting regions, an extracellular domain, a transmembrane domain, and an intracellular signaling domain. Optionally, the CAR comprises a hinge/transmembrane domain, as described herein. In such embodiments, the two or more antigen-specific targeting regions target at least two different antigens and may be arranged in tandem and separated by linker sequences. In another embodiment, the CAR is a bispecific CAR, which specific to two different antigens.

For example, a CAR as described herein is a bispecific CAR that can bind both CD37 and CD19. The CD37-binding site and CD19-binding site can each include an antibody reagent, e.g., a single chain variable fragment (scFv).

Accordingly, the CD37-binding sequence of the bispecific CAR is, in some embodiments, an antibody reagent. In other embodiments, the antibody reagent is an anti-CD37 scFv. In some embodiments, the VH of the anti-CD37 scFv corresponds to, comprises, or comprises a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 1. In some embodiments, the VL of the anti-CD37 scFv corresponds to, comprises, or comprises a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 2. The VH of the anti-CD37 scFv can be positioned N-terminal to the VL, or the VL can be positioned N-terminal to the VH. The VL and VH domains can optionally be connected via a linker, e.g., a linker of SEQ ID NO: 3. In some embodiments, the anti-CD37 scFv corresponds to the sequence of SEQ ID NO: 4 or 5; comprises the sequence of SEQ ID NO: 4 or 5; or comprises a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 4 or 5.

The CD19-binding sequence of the bispecific CAR is, in some embodiments, an antibody reagent. In other embodiments, the antibody reagent is an anti-CD19 scFv. In some embodiments, the VH of the anti-CD19 scFv corresponds to, comprises, or comprises a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 12. In some embodiments, the VL of the anti-CD19 scFv corresponds to, comprises, or comprises a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 13. The VH of the anti-CD19 scFv can be positioned N-terminal to the VL, or the VL can be positioned N-terminal to the VH. The VL and VH domains can optionally be connected via a linker, e.g., a linker of SEQ ID NO: 3. In some embodiments, the anti-CD19 scFv corresponds to the sequence of SEQ ID NO: 14; or comprises the sequence of SEQ ID NO: 14; or comprises a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 14.

Target/Antigen

Any cell surface moiety can be targeted by a CAR. Most often, the target will be a cell surface polypeptide differentially or preferentially expressed on a cell one wishes to target for a T cell response. In this regard, tumor antigens or tumor-associated antigens provide attractive targets, providing a means to target tumor cells while avoiding or at least limiting collateral damage to non-tumor cells or tissues.

As noted above, one target of the bispecific CAR is CD37. CD37 is cell surface protein that contains four hydrophobic transmembrane domains. CD37 is expressed exclusively on immune cells. It is highly expressed on mature B cells, and is moderately expressed on T cells and myeloid cells. CD37 sequences are known for a number of species, e.g., human CD37 (NCBI Gene ID: 951) polypeptide (e.g., NCBI Ref Seq NP_001035120.1) and mRNA (e.g., NCBI Ref Seq NM_001040031.1). CD37 can refer to human CD37, including naturally occurring variants, molecules, and alleles thereof. In some embodiments of any of the aspects, e.g., in veterinary applications, CD37 can refer to the CD37 of, e.g., dog, cat, cow, horse, pig, and the like. Homologs and/or orthologs of human CD37 are readily identified for such species by one of skill in the art, e.g., using the NCBI ortholog search function or searching available sequence data for a given species for sequence similar to a reference CD37 sequence.

The second target of the bispecific CAR is CD19. CD19 is a transmembrane protein expressed in all B lineage cells, except for plasma cells, and in follicular dendritic cells. CD19 sequences are known for a number of species, e.g., human CD19 (NCBI Gene ID: 930) polypeptide (e.g., NCBI GenBank Accession No.: AAB60697.1) and DNA (e.g., NCBI GenBank Accession No.: AH005421.2). CD19 can refer to human CD19, including naturally occurring variants, molecules, and alleles thereof. In some embodiments of any of the aspects, e.g., in veterinary applications, CD19 can refer to the CD19 of, e.g., cat, dog, cow, horse, pig, and the like. Homologs and/or orthologs of human CD19 are readily identified for such species by one of skill in the art, e.g., using the NCBI ortholog search function or searching available sequence data for a given species for sequence similar to a reference CD19 sequence.

Hinge and Transmembrane Domains

The binding domain of the CAR is optionally followed by one or more “hinge domains,” which plays a role in positioning the target binding domain away from the effector cell surface to enable proper cell/cell contact, target binding and activation. A CAR optionally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the bispecific CARs described herein include the hinge region derived from the extracellular regions of type 1 membrane proteins such as CD8 (e.g., CD8α), CD4, CD28, and CD7, which may be wild-type hinge regions from these molecules or may be altered. In one embodiment, the hinge domain comprises a CD8a hinge region.

CD8 is an antigen preferentially found on the cell surface of cytotoxic T lymphocytes. CD8 mediates cell-cell interactions within the immune system, and acts as a T cell co-receptor. CD8 consists of an alpha (CD8α or CD8a) and beta (CD8β or CD8b) chain. CD8a sequences are known for a number of species, e.g., human CD8a, (NCBI Gene ID: 925) polypeptide (e.g., NCBI Ref Seq NP_001139345.1) and mRNA (e.g., NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like. Homologs and/or orthologs of human CD8 are readily identified for such species by one of skill in the art, e.g., using the NCBI ortholog search function or searching available sequence data for a given species for sequence similar to a reference CD8 sequence.

As used herein, “transmembrane domain” (“TM domain”) refers to the portion of the CAR that fuses the extracellular binding portion, optionally via a hinge, to the intracellular portion (e.g., the intracellular signaling domain and the co-stimulatory domain, if present) and anchors the CAR to the plasma membrane of the immune effector cell. The transmembrane domain is a generally hydrophobic region of the CAR which crosses the plasma membrane of a cell. The transmembrane domain can be the transmembrane region or fragment thereof of a transmembrane protein (for example a Type I transmembrane protein or other transmembrane protein), an artificial hydrophobic sequence, or a combination thereof. While specific examples are provided herein and used in the Examples, other transmembrane domains will be apparent to those of skill in the art and can be used in connection with alternate embodiments of the technology. A selected transmembrane region or fragment thereof would preferably not interfere with the intended function of the CAR. As used in relation to a transmembrane domain of a protein or polypeptide, “fragment thereof” refers to a portion of a transmembrane domain that is sufficient to anchor or attach a protein to a cell surface.

In some examples, the transmembrane domain or fragment thereof of the CAR described herein comprises a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In one embodiment, the transmembrane domain or fragment thereof is derived from or comprises the transmembrane domain of CD8.

As used herein, a “hinge/transmembrane domain” refers to a domain comprising both a hinge domain and a transmembrane domain. In one embodiment, the hinge/transmembrane domain of a bispecific CAR or fragment thereof is derived from or comprises the hinge/transmembrane domain of CD8. The CD8 hinge/transmembrane domain can include the amino acid sequence of SEQ ID NO: 9, or variants thereof.

Co-Stimulatory Domains

The bispecific CARs described herein optionally comprise an intracellular domain of a co-stimulatory molecule, or co-stimulatory domain. As used herein, the term “co-stimulatory domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAGS), CD270 (HVEM), CD273 (PD-L2), CD274 (PD-L1), CD278 (ICOS), DAP10, LAT, NKD2C SLP76, TRIM, and ZAP70. For example, the intracellular domain is the intracellular domain of 4-1 BB.

4-1 BB is a membrane receptor protein, also known as CD137, which is a member of the tumor necrosis factor (TNF) receptor superfamily. 4-1 BB is expressed on activated T lymphocytes. 4-1 BB sequences are known for a number of species, e.g., human 4-1 BB, also known as TNFRSF9 (NCBI Gene ID: 3604) and mRNA (NCBI Reference Sequence: NM_001561.5). 4-1 BB can refer to human 4-1 BB, including naturally occurring variants, molecules, and alleles thereof. In some embodiments of any of the aspects, e.g., in veterinary applications, 4-1 BB can refer to the 4-1 BB of, e.g., dog, cat, cow, horse, pig, and the like. Homologs and/or orthologs of human 4-1 BB are readily identified for such species by one of skill in the art, e.g., using the NCBI ortholog search function or searching available sequence data for a given species for sequence similar to a reference 4-1 BB sequence.

For example, a 4-1 BB co-stimulatory domain of a bispecific CAR described herein can include the amino acid sequence of SEQ ID NO: 10, or variants thereof.

Intracellular Signaling Domain

The bispecific CARs as described herein comprise an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain. Non-limiting examples of immunoreceptor tyrosine-based activation motif (ITAM)-containing intracellular signaling domains that are of particular use in the technology include those derived from TCRζ, FcRγ, FcRβ, CD3γ, CD3θ, CD3δ, CD3ε, CD3ζ, CD22, CD79a, CD79b, and CD66d.

CD3 is a T cell co-receptor that facilitates T lymphocytes activation when simultaneously engaged with the appropriate co-stimulation (e.g., binding of a co-stimulatory molecule). A CD3 complex consists of 4 distinct chains; mammal CD3 consists of a CD3γ chain, a CD3δ chain, and two CD3εchains. These chains associate with a molecule known as the T cell receptor (TCR) and the CD3ζ to generate an activation signal in T lymphocytes. A complete TCR complex comprises a TCR, CD3ζ, and the complete CD3 complex.

In some embodiments of any aspect, a CAR polypeptide described herein comprises an intracellular signaling domain that comprises an immunoreceptor tyrosine-based activation motif (ITAM) from CD3 zeta (CD3ζ). In some embodiments of any aspect, the ITAM comprises three motifs of ITAM of CD3ζ (ITAM3). In some embodiments of any aspect, the three motifs of ITAM of CD3ζ are not mutated and, therefore, include native or wild-type sequences. For example, the CD3ζ sequence of a bispecific CAR described herein comprises the sequence of SEQ ID NO: 11, or variants thereof, as set forth below. In various embodiments, the CD3 sequence of such CAR polypeptides is a native or wild-type sequence.

A more detailed description of CARs and CART cells can be found in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

In some embodiments, the CAR further comprises a linker domain. As used herein “linker domain” or “linker region” refers to an oligo- or polypeptide region from about 2 to 100 amino acids in length, which links together any of the domains/regions of the CAR as described herein. In some embodiment, linkers can include or be composed of flexible residues such as glycine and serine so that the adjacent protein domains are free to move relative to one another, e.g., the linker of SEQ ID NO: 3. Longer linkers may be used when it is desirable to ensure that two adjacent domains do not sterically interfere with one another. Linkers may be cleavable or non-cleavable. Examples of cleavable linkers include 2A linkers (e.g., T2A), 2A-like linkers or functional equivalents thereof and combinations thereof. In some embodiments, the linker region is T2A derived from Thosea asigna virus. Non-limiting examples of linkers that can be used in this technology include P2A and F2A.

In some embodiments, a CAR as described herein further comprises a reporter molecule, e.g., to permit for non-invasive imaging (e.g., positron-emission tomography PET scan). In a bispecific CAR that includes a reporter molecule, the first extracellular binding domain and the second extracellular binding domain can include different or the same reporter molecule. In a bispecific CAR T cell, the first CAR and the second CAR can express different or the same reporter molecule. In another embodiment, a CAR as described herein further comprises a reporter molecule (for example hygromycin phosphotransferase (hph)) that can be imaged alone or in combination with a substrate or chemical (for example 9-[4-[¹⁸F]fluoro-3-(hydroxymethyl)butyl]guanine ([¹⁸F]FHBG)). In another embodiment, a CAR as described herein further comprises nanoparticles at can be readily imaged using non-invasive techniques (e.g., gold nanoparticles (GNP) functionalized with ⁶⁴Cu²⁺). Labeling of CAR T cells for non-invasive imaging is reviewed, for example in Bhatnagar et al., Integr Biol. (Camb). 5(1):231-238, 2013, and Keu et al., Sci Transl. Med. 18; 9(373), 2017, which are incorporated herein by reference in their entireties.

GFP and mCherry are demonstrated herein as fluorescent tags useful for imaging a CAR expressed on a T cell or NK cell (e.g., a CAR T cell or a CAR NK cell). It is expected that essentially any fluorescent protein known in the art can be used as a fluorescent tag for this purpose. For clinical applications, the CAR need not include a fluorescent tag or fluorescent protein.

In some embodiments, the CAR polypeptide sequence comprises the sequence of SEQ ID NO: 6, 7, 15, 16, 17, 18, 19, or 20. In some embodiments, the CAR polypeptide comprises a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 6, 7, 15, 16, 17, 18, 19, or 20.

### Nucleic Acids Encoding CARs

Also provided are nucleic acid constructs and vectors encoding the bispecific CAR polypeptides described herein for use in generating bispecific CAR T cells. In various examples, the invention provides constructs that each include separate coding sequences for multiple proteins to be expressed in a bispecific CAR T cell of the invention. These separate coding sequences can be separated from one another by a cleavable linker sequence as described herein. For example, sequences encoding viral 2A proteins (e.g., T2A) can be placed between the separate genes and, when transcribed, can direct cleavage of the generated polyprotein. As noted above, constructs and vectors of the invention can include any of a number of different combinations of sequences.

Furthermore, the polynucleotides of the invention can include the expression of a suicide gene. This can be done to facilitate external, drug-mediated control of administered cells. For example, by use of a suicide gene, modified cells can be depleted from the patient in case of, e.g., an adverse event. In one example, the FK506 binding domain is fused to the caspase9 pro-apoptotic molecule. T cells engineered in this manner are rendered sensitive to the immunosuppressive drug tacrolimus. Other examples of suicide genes are thymidine kinase (TK), CD20, thymidylate kinase, truncated prostate-specific membrane antigen (PSMA), truncated low affinity nerve growth factor receptor (LNGFR), truncated CD19, and modified Fas, which can be triggered for conditional ablation by the administration of specific molecules (e.g., ganciclovir to TK+ cells) or antibodies or antibody-drug conjugates.

Constructs including sequences encoding proteins for expression in the bispecific CAR T cells of the invention can be comprised within vectors. In various examples, the vectors are retroviral vectors. Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; each of which is incorporated by reference herein in its entirety. Lentiviral system for efficient DNA delivery can be purchased from OriGene; Rockville, Md. In various embodiments, the protein is expressed in the T or NK cell by transfection or electroporation of an expression vector comprising nucleic acid encoding the protein using vectors and methods that are known in the art. In some embodiments, the vector is a viral vector or a non-viral vector. In some embodiments, the viral vector is a retroviral vector (e.g., a lentiviral vector), an adenovirus vector, or an adeno-associated virus vector. In alternative embodiments, the CAR polypeptide of any of the bispecific CARs described herein are expressed in the mammalian cell via transfection or electroporation of an expression vector comprising nucleic acid encoding the CAR. Transfection or electroporation methods are known in the art.

Efficient expression of the bispecific CARs polypeptides in an immune cell as described herein can be assessed using standard assays that detect the mRNA, DNA, or gene product of the nucleic acid encoding the proteins. For example, RT-PCR, FACS, northern blotting, western blotting, ELISA, or immunohistochemistry can be used. The proteins described herein can be constitutively expressed or inducibly expressed. In some examples, the proteins are encoded by a recombinant nucleic acid sequence. In one embodiment, the CAR polypeptide described herein is constitutively expressed. In one embodiment, the CAR polypeptide described herein is encoded by recombinant nucleic acid sequence.

The invention also provides a composition that includes a vector that includes a polynucleotide sequence encoding a bispecific CAR comprising an extracellular domain comprising a sequence that specifically binds to CD37 and CD19, as described herein.

For example, the invention provides a nucleic acid capable of encoding the CAR polypeptide of SEQ ID NO: 6, 7, 15, 16, 17, 18, 19, or 20, or a CAR polypeptide comprising a sequence with at least 80%, at least 85%, at least 90%, at least 91%, at least 92%, at least 93%, at least 94%, at least 95%, at least 96%, at least 97%, at least 98%, at least 99% or greater sequence identity to the sequence of SEQ ID NO: 6, 7, 15, 16, 17, 18, 19, or 20.

### Immune Cells

One aspect of the technology relates to an immune cell comprising any of the bispecific CAR polypeptides described herein (e.g., SEQ ID NO: 15, 16, 19, or 20); or a nucleic acid encoding any of the bispecific CAR polypeptides described herein. In one embodiment, the immune cell comprises an antibody, antibody reagent, antigen-binding portion thereof, or any of the bispecific CARs described herein, or a nucleic acid encoding such an antibody, antibody reagent, antigen-binding portion thereof, or any of the bispecific CARs described herein. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. The immune cell can be a T cell; a NK cell; a NKT cell; lymphocytes, such as B cells and T cells; and myeloid cells, such as monocytes, macrophages, eosinophils, mast cells, basophils, and granulocytes. In some embodiments, the immune cell is a T cell. In other embodiments, the immune cell is an NK cell.

Immune cells (e.g., human immune cells) that can be used in the invention include autologous cells, obtained from the subject to whom the cells are later to be administered, after ex vivo modification and expansion. For example, the immune cells can be obtained from an individual having or diagnosed as having cancer, an autoimmune disease, or a plasma cell disorder. Immune cells can also be obtained from allogeneic donors, which are non-genetically identical individuals of the same species as the intended recipients of the cells. Immune cells useful for the invention include T cells and natural killer (NK) cells.

Methods for obtaining T cells and NK are known in the art and can be useful for the engineered immune cells described herein. T cells and NK cells are typically obtained from peripheral blood that is collected from a subject by, e.g., venipuncture or withdrawal through an implanted port or catheter. Optionally, the blood can be obtained by a process including leukapheresis, in which white cells are obtained from the blood of a subject, while other blood components are returned to the subject. Blood or leukapheresis product (fresh or cryopreserved) is processed to enrich for T cells or NK cells using methods known in the art. For example, density gradient centrifugation (using, e.g., Ficoll) and/or counter-flow centrifugal elutriation can be carried out to enrich for mononuclear cells (including T cells or NK cells). In one example, for T cells, a T cell stimulation step employing, e.g., CD3/CD28 antibodies coated on magnetic beads or artificial antigen presenting cells (aAPCs) expressing, e.g., cell surface-bound anti-CD3 and anti-CD28 antibody fragments (see below), can further be carried out in order to stimulate T cells and to deplete other cells, e.g., B cells. The T cells of enriched T cell preparations can then be subject to genetic modification.

As an alternative to peripheral blood, tissues including bone marrow, lymph nodes, spleen, and tumors can be used as a source for T cells and NK cells. The T cells and NK cells can be of human, primate, hamster, rabbit, rodent, cow, pig, sheep, horse, goat, dog, or cat origin, but any other mammalian cell may be used. In a certain embodiments of any aspect, the T cells and NK cells cell is human.

An immune cell, e.g., a T cell or NK cell, can be engineered to comprise any of the bispecific CAR polypeptides described herein (e.g., SEQ ID NO: 15, 16, 19, or 20); or a nucleic acid encoding any of the CAR polypeptides described herein. In some embodiments, the any of the bispecific CAR polypeptides described herein are comprised in a lentiviral vector. The lentiviral vector is used to express the CAR polypeptide in a cell using infection standard techniques. In some embodiments, the immune cell (e.g., a T cell or NK cell) is obtained from an individual having or diagnosed as having a cancer expressing CD37. In some embodiments, the immune cell is obtained from an individual non-responsive to and/or concurrently receiving anti-CD19 and/or anti-CD20 therapy.

### Therapeutic Methods

The invention provides methods and compositions for use in treating and preventing diseases and conditions including, for example, cancer, autoimmune diseases or disorders, or plasma cell diseases or disorders. These methods include the use of an immune cell (e.g., a T cell or an NK cell) including a bispecific CAR as described herein, and administering the modified immune cell to a subject to treat, e.g., cancer. In some embodiments of any of the aspect, the modified immune cell (e.g., a T cell or an NK cell including one or more additional modification as described herein) is stimulated and/or activated prior to administration to the subject.

As used herein, a “condition” includes cancer, an infectious disease, an autoimmune disease or disorder, a plasma cell disease or disorder, or a condition relating to transplantation. Subjects having a disease or condition can be identified by a physician using current methods of diagnosing the disease or condition. Symptoms and/or complications of the disease or condition, which characterize these conditions and aid in diagnosis are well known in the art and include, but are not limited to, fatigue, persistent infections, and persistent bleeding. Tests that may aid in a diagnosis of, e.g., the disease or condition include, but are not limited to, blood screening and bone marrow testing, and are known in the art for a given condition. A family history for a disease or condition, or exposure to risk factors for a disease or condition, can also aid in determining if a subject is likely to have the disease or condition or in making a diagnosis of the disease or condition.

“Cancer” as used herein can refer to a hyperproliferation of cells whose unique trait, loss of normal cellular control, results in unregulated growth, lack of differentiation, local tissue invasion, and metastasis, and can be a leukemia, a lymphoma, multiple myeloma, or a solid tumor. Non-limiting examples of leukemias include acute myeloid leukemia (AML), chronic myeloid leukemia (CML), acute lymphocytic leukemia (ALL), and chronic lymphocytic leukemia (CLL). In one embodiment, the leukemia is CLL. Non-limiting examples of lymphoma include B cell non-Hodgkin lymphoma (NHL), diffuse large B-cell lymphoma (DLBCL), follicular lymphoma (FL), small lymphocytic lymphoma (SLL), mantle cell lymphoma (MCL), marginal zone lymphomas, Burkitt's lymphoma, hairy cell leukemia (HCL), T cell lymphoma, peripheral T cell lymphoma (PTCL), cutaneous T cell lymphoma (CTCL), angioimmunoblastic T cell lymphoma (AITL), and anaplastic large cell lymphoma (ALCL)). In one embodiment, the cancer is MCL, DLBCL, FL, Burkitt's lymphoma, PTCL, CTCL, AITL, or ALCL. Non-limiting examples of solid tumors include adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), giant cell tumor of bone and soft tissue, glioblastoma, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, and Wilms tumor. Solid tumors can be found in bones, muscles, or organs, and can be sarcomas or carcinomas. It is contemplated that any aspect of the technology described herein can be used to treat all types of cancers, including cancers not listed in the instant application. As used herein, the term “tumor” refers to an abnormal growth of cells or tissues, e.g., of malignant type or benign type.

As used herein, an “autoimmune disease” or “autoimmune disorder” is characterized by the inability of one's immune system to distinguish between a foreign cell and a healthy cell. This results in one's immune system targeting one's healthy cells for programmed cell death. Non-limiting examples of an autoimmune disease or disorder include inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity. Other examples of auto-immune-related disease or disorder, but should not be construed to be limited to, include rheumatoid arthritis, multiple sclerosis (MS), systemic lupus erythematosus, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, acute disseminated encephalomyelitis, antiphospholipid antibody syndrome, aplastic anemia, idiopathic thrombocytopenic purpura, myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

A plasma cell is a white blood cell produces from B lymphocytes which function to generate and release antibodies needed to fight infections. As used herein, a “plasma cell disorder or disease” is characterized by abnormal multiplication of a plasma cell. Abnormal plasma cells are capable of “crowding out” healthy plasma cells, which results in a decreased capacity to fight a foreign object, such as a virus or bacterial cell. Non-limiting examples of plasma cell disorders include amyloidosis, Waldenstrom's macroglobulinemia, osteosclerotic myeloma (POEMS syndrome), monoclonal gammopathy of unknown significance (MGUS), and plasma cell myeloma.

Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu et al., Pharmacological Reviews 52:493-512, 2000; which are all incorporated by reference herein in their entireties. Lentiviral system for efficient DNA delivery can be purchased from OriGene; Rockville, Md.

One aspect of the technology described herein relates to a method of treating a cancer in a subject in need thereof, the method comprising: administering the cell of any of the mammalian cells comprising the any of the CAR polypeptides described herein.

Cluster of differentiation (CD) molecules are cell surface markers present on leukocytes. As a leukocyte differentiates and matures its CD profile changes. In the case that a leukocytes turns into a cancer cell, (i.e., a lymphoma), its CD profile is important in diagnosing the disease. The treatment and prognosis of certain types of cancers is reliant on determining the CD profile of the cancer cell. “CDX+”, wherein “X” is a CD marker, indicates the CD marker is present in the cancer cell, while “CDX−” indicates the marker is not present. One skilled in the art will be capable of assessing the CD molecules present on a cancer cell using standard techniques, for example using immunofluorescence to detect commercially available antibodies bound to the CD molecules.

In some embodiments, the cancer expresses one or more CD molecules. The bispecific CARs described herein can be used to treat a cancer that expresses CD37. In some embodiments, the CD37+ cancer is a lymphoma or a leukemia. For example, the lymphoma is B-cell non-Hodgkin lymphoma (NHL) (e.g., mantle cell lymphoma (MCL), Burkitt's lymphoma, diffuse large B cell lymphoma (DLBCL), follicular lymphoma, or Burkitt's lymphoma) or a T cell lymphoma (e.g., peripheral T cell lymphoma (PTCL), cutaneous T cell lymphoma (CTCL), angioimmunoblastic T cell lymphoma (AITL), or anaplastic large cell lymphoma (ALCL)). In another example, the leukemia is chronic lymphocytic leukemia (CLL).

Furthermore, cancer cells can evolve in response to treatment to alter its CD profile in order to evade said treatment. For example, a patient with a CD19+ leukemia or lymphoma can be treated with an anti-CD19 therapy. Following treatment, the cancer cell can relapse, or return after treatment, and no longer express the CD19 marker, resulting in a CD19− leukemia or lymphoma. As a result, the cancer will no longer be targetable by an anti-CD19 therapy.

Provided are methods using immune cells comprising a bispecific CAR as described herein (e.g., a CAR targeting CD37 and CD19) to treat a cancer in a subject that is non-responsive, or refractory to anti-CD19 and/or anti-CD20 therapy. In some embodiments, the immune cells comprising a bispecific CAR as described herein (e.g., a CAR targeting CD37 and CD19) are used to treat a cancer in a subject having a cancer that is CD19- and/or CD20-. In some embodiments, the immune cells comprising a bispecific CAR as described herein (e.g., a CAR targeting CD37 and CD19) are used to treat a cancer in a subject having a cancer that is relapsed and no longer expresses CD19 or CD20.

Additionally, immune cells comprising a bispecific CAR as described herein (e.g., a CAR targeting CD37 and CD19) can be used to treat a cancer in a subject in need thereof, wherein the subject is concurrently administered anti-CD19 and/or anti-CD20 therapy.

In some embodiments of any of the aspect, the immune cell (e.g., T cell or NK cell) comprising a bispecific CAR is stimulated and/or activated prior to administration to the subject.

### Administration

In some embodiments, the methods described herein relate to treating a subject having or diagnosed as having cancer, a plasma cell disease or disorder, or an autoimmune disease or disorder with a mammalian cell comprising any of the CAR polypeptides described herein, or a nucleic acid encoding any of the CAR polypeptides described herein. A bispecific CAR T or NK cells as used herein refers to a mammalian T or NK cell comprising any of the bispecific CAR polypeptides as described, or a nucleic acid encoding any of the bispecific CAR polypeptides.

The compositions described herein can be administered to a subject having or diagnosed as having a condition. In some embodiments, the methods described herein comprise administering an effective amount of bispecific CAR T or NK cells described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In one embodiment, the compositions described herein are administered systemically or locally. In a preferred embodiment, the compositions described herein are administered intravenously. In another embodiment, the compositions described herein are administered at the site of a tumor.

The term “effective amount” as used herein refers to the amount of bispecific CAR T or NK cells needed to alleviate at least one or more symptom of the disease or disorder, and relates to a sufficient amount of the cell preparation or composition to provide the desired effect. The term “therapeutically effective amount” therefore refers to an amount of bispecific CAR T or NK cells that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of bispecific CAR T or NK cells, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

In one aspect of the technology, the technology described herein relates to a pharmaceutical composition comprising bispecific CAR T or NK cells as described herein, and optionally a pharmaceutically acceptable carrier. The active ingredients of the pharmaceutical composition at a minimum comprise bispecific CAR T or NK cells as described herein. In some embodiments, the active ingredients of the pharmaceutical composition consist essentially of bispecific CAR T or NK cells as described herein. In some embodiments, the active ingredients of the pharmaceutical composition consist of bispecific CAR T or NK cells as described herein. Pharmaceutically acceptable carriers for cell-based therapeutic formulation include saline and aqueous buffer solutions, Ringer's solution, and serum component, such as serum albumin, HDL and LDL. The terms such as “excipient,” “carrier,” “pharmaceutically acceptable carrier” or the like are used interchangeably herein.

In some embodiments, the pharmaceutical composition comprising bispecific CAR T or NK cells as described herein can be a parenteral dose form. Since administration of parenteral dosage forms typically bypasses the patient's natural defenses against contaminants, the components apart from the bispecific CAR T or NK cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the bispecific CAR T or NK cells preparation prior to administration.

Suitable vehicles that can be used to provide parenteral dosage forms of bispecific CAR T or NK cells as disclosed within are well known to those skilled in the art. Examples include, without limitation: saline solution; glucose solution; aqueous vehicles including but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Dosage

“Unit dosage form” as the term is used herein refers to a dosage for suitable one administration. By way of example, a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

In some embodiments, the bispecific CAR T or NK cells T cells described herein are administered as a monotherapy, i.e., another treatment for the condition is not concurrently administered to the subject. A pharmaceutical composition comprising the T or NK cells described herein can generally be administered at a dosage of 10⁴ to 10⁹ cells/kg body weight, in some instances 10⁵ to 10⁶ cells/kg body weight, including all integer values within those ranges. If necessary, T or NK cell compositions can also be administered multiple times at these dosages. The cells can be administered by using infusion techniques that are commonly known in immunotherapy (see, e.g., Rosenberg et al., New Eng. J. Med. 319:1676, 1988).

In certain aspects, it may be desired to administer bispecific CAR T or NK cells to a subject and then subsequently redraw blood (or have an apheresis performed), activate the T or NK cells therefrom as described herein, and reinfuse the patient with these activated and expanded T or NK cells. This process can be carried out multiple times every few weeks. In certain aspects, T or NK cells can be activated from blood draws of from 10 cc to 400 cc. In certain aspects, T or NK cells are activated from blood draws of 20 cc, 30 cc, 40 cc, 50 cc, 60 cc, 70 cc, 80 cc, 90 cc, or 100 cc.

Modes of administration can include, for example intravenous (i.v.) injection or infusion. The compositions described herein can be administered to a patient transarterially, intratumorally, intranodally, or intramedullary. In some embodiments, the compositions of T or NK cells may be injected directly into a tumor, lymph node, or site of infection. In one embodiment, the compositions described herein are administered into a body cavity or body fluid (e.g., ascites, pleural fluid, peritoneal fluid, or cerebrospinal fluid).

In a particular exemplary aspect, subjects may undergo leukapheresis, wherein leukocytes are collected, enriched, or depleted ex vivo to select and/or isolate the immune cells of interest, e.g., T cells or NK cells. These immune cell isolates can be expanded e.g., in the case of T cells, by contact with an aAPC as described herein, such as an aAPC expressing anti-CD28 and anti-CD3 CDRs, and treated such that one or more CAR constructs of the technology may be introduced, thereby creating a CAR T cell. Subjects in need thereof can subsequently undergo standard treatment with high dose chemotherapy followed by peripheral blood stem cell transplantation. Following or concurrent with the transplant, subjects can receive an infusion of the expanded CAR T cells or NK cells. In one embodiment, expanded cells are administered before or following surgery.

In some embodiments, lymphodepletion is performed on a subject prior to administering one or more CAR T or NK cell as described herein. In such embodiments, the lymphodepletion can comprise administering one or more of melphalan, cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combinational Therapy

The bispecific CAR T or NK cells described herein can be used in combination with other known agents and therapies. For example, the subject can be further administered an anti-CD19 therapy and/or an anti-CD20 therapy. In one embodiment, the subject is resistant to anti-CD19 and/or anti-CD20 therapies. In another embodiment, the subject is concurrently administered and anti-CD19 and/or anti-CD20 therapy.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The bispecific CAR T or NK cells described herein and the at least one additional therapeutic agent can be administered simultaneously, in the same or in separate compositions, or sequentially. For sequential administration, the CAR-expressing immune cell described herein can be administered first, and the additional agent can be administered second, or the order of administration can be reversed. The bispecific CAR T or NK therapy and/or other therapeutic agents, procedures or modalities can be administered during periods of active disorder, or during a period of remission or less active disease. The CAR T or NK therapy can be administered before another treatment, concurrently with the treatment, post-treatment, or during remission of the disorder.

When administered in combination, the bispecific CAR T or NK cells and the additional agent (e.g., second or third agent), or all, can be administered in an amount or dose that is higher, lower or the same as the amount or dosage of each agent used individually, e.g., as a monotherapy. In certain embodiments, the administered amount or dosage of the bispecific CAR T or NK cells, the additional agent (e.g., second or third agent), or all, is lower (e.g., at least 20%, at least 30%, at least 40%, or at least 50%) than the amount or dosage of each agent used individually. In other embodiments, the amount or dosage of the bispecific CAR T or NK cells, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect. In further embodiments, the bispecific CAR T or NK cells described herein can be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, rapamycin, mycophenolic acid, steroids, FR901228, cytokines, or a peptide vaccine, such as that described in Izumoto et al., J. Neurosurg. 108:963-971, 2008.

For example, the bispecific CAR T or NK cells described herein can be used in combination with an anti-CD19 therapy. Examples of anti-CD19 therapies include, but are not limited to, blinatumomab, coltuximabravtansine, MOR208, MEDI-551, denintuzumabmafodotin, taplitumomabpaptox, XmAb 5871, MDX-1342, AFM11, DI-B4, axicabtagene ciloleucel, and tisagenlecleucel. Furthermore, the bispecific CAR T or NK cells described herein can be used in combination with an anti-CD20 therapy. Examples of anti-CD20 therapies include, but are not limited to, rituximab, ocrelizumab, obinutuzumab, ofatumumab, ibritumomab, tiuxetan, tositumomab, ublituximab, ocaratuzumab, IMMU-106, and GA-101.

In further embodiments, the bispecific CAR T or NK cells described herein can be used in combination with a checkpoint inhibitor. Exemplary checkpoint inhibitors include anti-PD-1 inhibitors (nivolumab, MK-3475, pembrolizumab, pidilizumab, AMP-224, AMP-514), anti-CTLA-4 inhibitors (ipilimumab and tremelimumab), anti-PD-L1 inhibitors (atezolizumab, avelomab, durvalumab, MSB0010718C, MED14736, and MPDL3280A), and anti-TIM3 inhibitors.

In further embodiments, the bispecific CAR T or NK cells described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), ibrutinib, (IMBRUVICA®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amadei®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (IR,2R,45)-4-[(2R)-2 [(1R,95,125,15R,16E,18R,19R,21R,235,24E,26E,28Z,305,325,35R)-I,18-dihydroxy-19,30-dimethoxy-15,17,21,23, 29,35-hexamethyl-2,3,10,14,20-pentaoxo-11,36-dioxa-4-azatricyclo[30.3.1.04′9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOI); rapamycin (AY22989, Sirolimus®); simapimod (CAS 164301-51-3); emsirolimus, (5-{2,4-Bis[(35,)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-I-(((5)-4-methyl-I-((R)-2-methyloxiran-2-yl)-I-oxopentan-2-yl)amino)-I-oxo-3-phenylpropan-2-yl)-2-((5,)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-RIIS′)-2-[(2R)-2-methyl-2-oxiranyl]-2-oxo-I-(phenylmethyl)ethyl]-L-serinamide (ONX-0912).

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18^{th} edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In an embodiment, bispecific CAR T or NK cells described herein are administered to a subject in combination with a molecule that decreases the level and/or activity of a molecule targeting GITR and/or modulating GITR functions, a molecule that decreases the Treg cell population, an mTOR inhibitor, a GITR agonist, a kinase inhibitor, a non-receptor tyrosine kinase inhibitor, a CDK4 inhibitor, and/or a BTK inhibitor.

### Efficacy

The efficacy of bispecific CAR T or NK cells in, e.g. the treatment of a condition described herein, or to induce a response as described herein (e.g. a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein.

Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein, for example treatment of a cancer. When using an experimental animal model, efficacy of treatment is evidenced when a statistically significant change in a marker is observed.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

### EXAMPLES

The following are examples of the methods and compositions of the invention. It is understood that various other embodiments may be practiced, given the description provided herein. It is noted that Examples 1-11 provide the materials and methods used for the experiments described herein, while Examples 12-19 demonstrate results and Example 20 provides sequences.

### Example 1. Primary Human T Cell Culture

For primary T lymphocyte expansions, bulk human T cells were activated (day 0) using anti-CD3/CD28 Dynabeads (LifeTechnologies), followed by transduction with a lentiviral vector encoding the CAR 24-hours later. T cells were cultured in media supplemented with 20 IU/ml rhIL-2 beginning on day 0 of culture and were maintained at a constant cell concentration of 0.5×10⁶/mL by counting every 2-3 days. For functional assays, CAR T cells were cryopreserved at day 8-10 of culture, and upon thawing, were immediately stimulated with antigen or injected into mice.

### Example 2. Cell Lines and Culture Conditions

The JEKO-1, RAJI and wild-type parental K562 cells were purchased from American Type Culture Collection (ATCC). K562 cells were engineered to express CD37 and CD19 (K562-CD37-CD19). For some assays, cell lines were engineered to constitutively express click beetle green (CBG) luciferase/enhanced GFP (eGFP) and then sorted on a FACSAria (BD Biosciences®) to obtain a 99% pure population (CBG-GFP+). The cell lines were cultured in RPMI media containing 10% fetal bovine serum (FBS), penicillin, and streptomycin.

### Example 3. Flow Cytometry

The following antibodies were used: CD37-APC (clone MB-1, eBioscience®), CD37-BV711 (clone MB-371, BD Biosciences®), CD19-Pacific Blue (clone HIB19, Biolegend®), CD19-FITC (clone 4G7, BD), CD5-BUV737 (clone UCHT2, BD), CD20-APC Cy7 (clone 2H7, Biolegend®), CD79b-PE (clone CB3-1, eBioscience®), CD3-BV786 (clone SK7, BD), CD3-BV605 (clone OKT3, Biolegend®), CD45-PeCy7 (clone H130, Biolegend®), CD16-PE (clone B73.1, Biolegend®), CD14-Pacific Blue (clone HCD14, Biologend®), CD56-APC (clone HCD56, Biolegend®), CD33-BV510 (clone P67.6, Biolegend®), CD107a-AF700 (clone H4A3, BD Biosciences®), CD69-APC (clone FN50, Biolegend®), and IFNγ-FITC (clone GZ-4, eBioscience®). Cells were stained for 30 min in the dark at 4° C. and washed twice in PBS with 2% FBS. DAPI was added to gate on viable cells before acquisition. Antigen density was measured using antibodies bound per cell (ABC) and was calculated using Quantum™ Simply Cellular (Bangs Laboratories).

### Example 4. Fratricide Assay

Human T cells purified from anonymous human healthy donor leukopaks were activated with Cell Stimulation Cocktail (eBioscience®, Catalog #00-4970-03) for 6 hours. Activated and non-activated T cells were labelled with CFSE (ThermoFisher®, Catalog #C34554) following manufacturer's instructions and co-cultured with CAR-37, CAR-19 or untransduced T cells generated form the same normal donors. After 24 hours, flow cytometry was used to count the number of CFSE positive cells in each condition.

### Example 5. Immune Cell Isolation, Differentiation, and Co-Culture Assay

PBMCs from three normal donors were isolated with Ficoll-Paque PLUS (GE Healthcare, C987R36) and monocytes were purified with StemCell® kit (Catalog #19359). M1/M2 macrophages were generated in vitro as previously described (Zhang et al., PLoS One. 11(4):e0153550, 2016). Monocytes, macrophages, NK, and T cells were cultured at 1:1 E:T ratio with CAR-37 CAR-19 or untransduced T cells for 6 hours and CD107a and IFNγ production was measured by flow cytometry. PMA/ionomycin was used as positive control. Values were normalized on media and graphs represent fold change.

### Example 6. Cellular Cytotoxicity and Cytokines Assay

For cytotoxicity assays, CAR T cell effector cells were co-cultured with CBG luciferase-expressing tumor targets at the indicated ratios for 16 hours. Luciferase activity was measured with a Synergy Neo2 luminescence microplate reader (Biotek®). For the analysis of soluble cytokines, effector cells were co-cultured with tumor targets at a 1:1 ratio for 24 hours.

CAR-37 and CAR-19 T cells were normalized for CAR expression by adding untransduced but cultured and activated T cells from the same donor to achieve the same proportion of CAR+ T cells in each sample. For cytotoxicity assays, percent specific lysis was calculated by the following equation: % specific lysis=(total RLU/target cells only RLU)×100. For cytokine assays, cell-free supernatants were analyzed for cytokine expression using a Luminex array (Luminex Corp, FLEXMAP 3D®) according to the manufacturer's instructions. All samples were measured in technical duplicates. Duplicates were averaged before graphing with GraphPad Prism® 7 (version 7.0). In addition, all assays were performed with biologic duplicates or triplicates or more, as indicated by the N in each experiment, which is based on the number of unique healthy donors T cells tested.

### Example 7. Jurkat Reporter Activation Assay

Jurkat (NFAT-Luc) reporter cells (Signosis, SL-0032) were transduced with the different CAR constructs. They were co-cultured at 1:1 E:T ratio for 24 hours with B cell lymphoma tumor cells or Nalm6 leukemia cells; anti-CD3/CD28 beads were used as positive control and media as negative control. Luciferase activity was measured after 16 hours with a Synergy Neo2 luminescence microplate reader (Biotek®). Relative activation was calculated on PMA.

### Example 8. Immunohistochemistry

Paraffin sections were deparaffinized with xylene and then rehydrated with a series of ethanol washes followed by H₂O. Antigen retrieval was conducted by microwaving slides for 15 min in 0.01 M sodium citrate buffer, pH 6.0. After washing with phosphate-buffered saline containing 0.1% Tween-20 (PBS-T), endogenous peroxidase activity was quenched with 3% H₂O₂ for 10 min. Slides were then washed again with PBS-T and blocked with Novolink Protein Block for 30 min at 25° C. After additional washing with PBS-T, slides were incubated with PBS-T containing 5% goat serum and mouse anti-CD37 (Invitrogen®, Catalog #MA5-15492) diluted 1:150 for 1 hr at 25° C. Following washing with PBS-T, slides were incubated with Cell Signaling Technology Signal Stain Boost IHC murine detection reagent for 30 min at 25° C., washed again with PBS-T, and incubated with DAB diluent (Vector Labs) containing DAB chromogen. After stain development, slides were again washed with PBS-T and counterstained with hematoxylin.

### Example 9. TMA Construction

Formalin-fixed paraffin embedded tissues involved by peripheral T cell lymphoma (PTCL) were retrieved from the archives of the Department of Pathology at Brigham and Women's Hospital. Cores (0.6 mm in diameter) from donor blocks were transferred to recipient blocks to create a tissue microarray in the Tissue Microarray Core of the Dana Farber/Harvard Cancer Center and used to prepare 4 micron sections for immunohistochemical staining studies.

### Example 10. In Vivo Studies

NOD-SCID-γ chain−/− (NSG) (The Jackson Laboratory®) were engrafted with JEKO-1 cell line or patient derived tumor cells via the route of administration described. Cryopreserved CAR-37, CAR-19, or untransduced T cells were injected intravenously after engraftment of tumor was confirmed by luminescence. Tumor burden was regularly monitored using an Ami spectral imaging apparatus and analyzed with IDL software v. 4.3.1 following an intraperitoneal injection of D-Luciferin substrate solution (30 mg/mL). Animals were euthanized as per the experimental protocol or when they met pre-specified endpoints defined by the IACUC.

### Example 11. Statistical Analysis

Unless otherwise stated, a 2-tailed Student t test or 2-way Anova test were used for normal data at equal variance. Significance was considered for P<0.05. Analyses were performed with GraphPad Prism® 7 (version 7.0).

### Example 12. Construction of CARs and T Cell Culture Transduction

Two anti-CD37 CAR constructs were synthesized and cloned into a third-generation lentiviral plasmid backbone under the regulation of a human EF-1α promoter. All the CARs bear a CD8 hinge, 4-1 BB costimulatory domain and CD3ζ signaling domain. Vectors also contained a second transgene coding for the fluorescent reporter mCherry to facilitate enumeration of transduction efficiency. Human T cells were purified (StemCell Technologies®, Catalog #15061) from anonymous human healthy donor leukopacs purchased from the MGH blood bank under an IND-exempt protocol. Details of T cell culture are provided in Example 1.

### Example 13. CD37 is Highly Expressed on Human Lymphoma

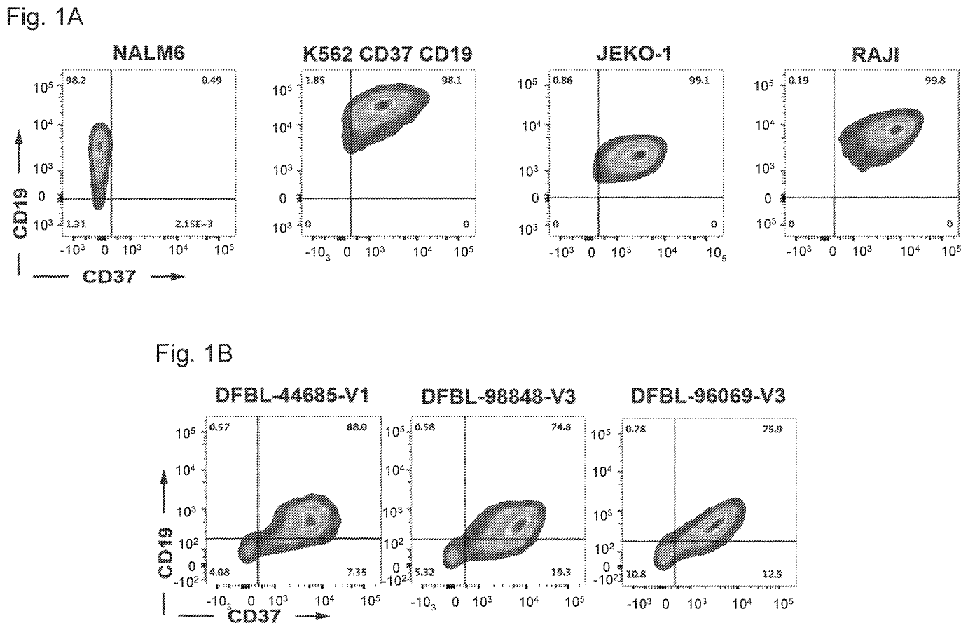

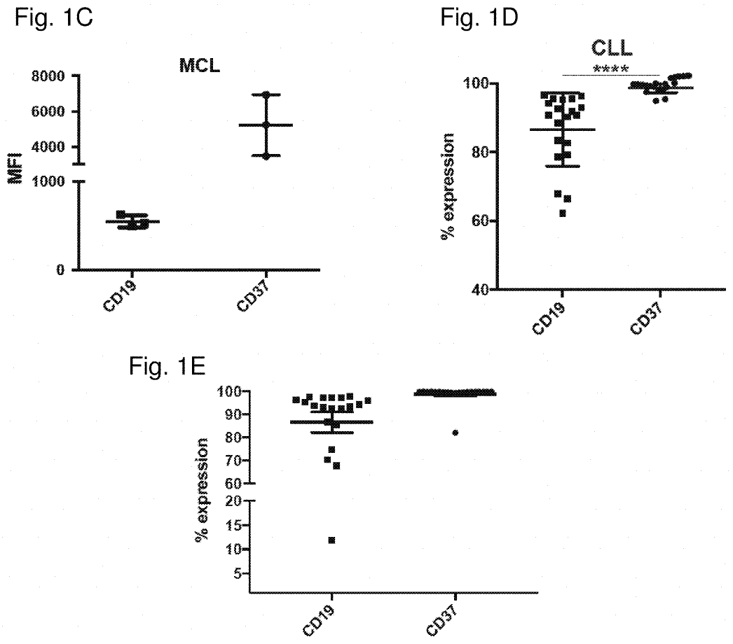

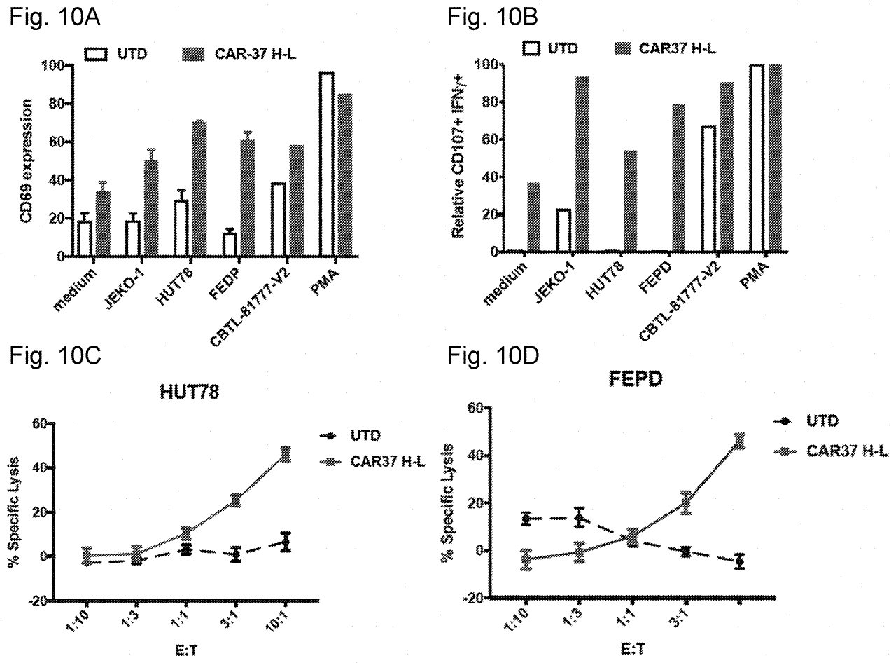

Flow cytometry was used to examine CD37 and CD19 expression on the leukemia and non-Hodgkin's lymphoma cell lines Nalm6, Jeko-1 and Raji (FIG. 1A) and in patient-derived MCL lines (FIGS. 1B and 1C) and primary patient CLL cells (FIG. 1D). Also generated were K562 cells transduced with both CD37 and CD19 to use as artificial antigen presenting cells for in vitro stimulation and a positive control for cytotoxicity assays (FIG. 1A). Although pre-B cell-derived leukemia cells (Nalm6) expressed CD19 but not CD37 (FIG. 1A), all the lymphoma cells expressed both CD19 and CD37 (FIGS. 1A-1C). In the patient-derived MCL samples, high and uniform expression of CD37 was noted, even higher than CD19, based on mean fluorescence intensity of the gated positive cells (FIG. 10), though it is recognized that this difference could reflect antibody binding differences or brightness of the fluorophores.

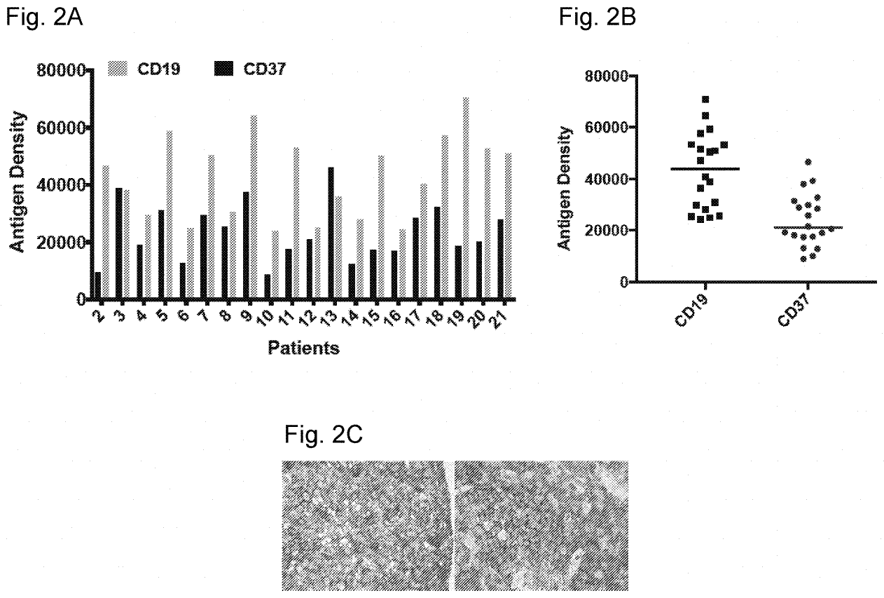

Next, CD37 and CD19 expression was evaluated on PBMC derived from 21 patients with chronic lymphocytic leukemia by flow cytometry (FIG. 1D), and again noted higher and more uniform expression of CD37 among the CD3-negative lymphocytes compared to CD19. When gated on the CD3-CD20+ B cells, CD37 expression remained high and uniform (FIG. 1E). To determine the level of antigen density for both antigens, the expression of CD19 and CD37 on these 21 samples was quantified using beads (FIG. 2A). It was observed that antigen density on a per-cell basis was higher for CD19 than for CD37 (mean=31,829±3,212 antibodies bound per cell (ABC) for CD19, vs 29,680±3232 ABC for CD37) (FIG. 2B, Table 1).

TABLE 1

| CLL PBMC # | CD19+ | CD37+ | CD5+ | CD20+ | CD79b+ |
|---|---|---|---|---|---|
| 2 | 95.6 | 98.9 | 73.3 | 87.6 | 13.3 |
| 3 | 90.7 | 99.7 | 94.4 | 91.3 | 11.4 |
| 4 | 79.2 | 99.9 | 44.7 | 96.8 | 59.7 |
| 5 | 94.2 | 99.8 | 84.5 | 87.1 | 6.09 |
| 6 | 67.9 | 99.3 | 49.8 | 63.7 | 10.5 |
| 7 | 88.4 | 97.6 | 15.3 | 91.2 | 11.5 |
| 8 | 82.7 | 99.6 | 93.2 | 95.2 | 27.8 |
| 9 | 90.7 | 99.4 | 2.22 | 97.3 | 10.5 |
| 10 | 78.6 | 94.9 | 85.6 | 18 | 10.6 |
| 11 | 95.2 | 97.6 | 96.3 | 95.2 | 11.7 |
| 12 | 90.2 | 99.6 | 73.6 | 85.4 | 31.4 |
| 13 | 92.6 | 99.7 | 92.5 | 81.8 | 29.1 |
| 14 | 62.3 | 99.1 | 54.6 | 94.8 | 41.7 |
| 15 | 91.8 | 99.2 | 93.2 | 98.8 | 39.6 |
| 16 | 66.4 | 99.5 | 38.3 | 75.3 | 18.1 |
| 17 | 96.3 | 99.7 | 95.5 | 96.6 | 42.9 |
| 18 | 95.6 | 97.9 | 80.9 | 97.6 | 65.8 |
| 19 | 92.9 | 97.8 | 93.8 | 65 | 11.7 |
| 20 | 83.4 | 95.4 | 84.6 | 92.3 | 12.6 |
| 21 | 96.6 | 99.1 | 89.7 | 90.5 | 7.14 |

Based on 29 samples of bone marrow aspirates, lymph node biopsies, and peripheral blood from patients with hematologic malignancies, expression of CD37 was confirmed on B cell lymphomas and normal B cells, and not on hematopoietic stem cells from normal donors or hematogones (Table 2).

TABLE 2

| | Clinical Findings From | CD37 | | |
|---|---|---|---|---|
| Specimen Type | Path Report | Expression | Immunophenotype | Other Notes |
| PBL | B-Cell Lymphoma: Splenic | + | B cells: CD19+ CD20+ | Asymptomatic, non-bulky |
| | Marginal Zone Lymphoma | | CD5−/dim CD10− CD23− | disease with a modestly |
| | | | CD38− CD200dim CD25− | enlarged spleen (17.5 cm), |
| | | | CD11c−/+ CD103− with | referred for evaluation in |
| | | | monotypic moderate surface | the context of leukocytosis |
| | | | lambda immunoglobulin | up to 29.97 K/uL at time of |
| | | | light chain expression | testing. |
| | | | | No prior lines of therapy. |
| BMA | Myelofibrosis: primary | − | Myeloid blasts: CD33+ | *Normal B cells +, |
| | myelofibrosis vs post- | | CD13− MPO− CD117+ | patient actively on JAK2 |
| | essential | | CD34+ HLA-DR+ | inhibitor at time of |
| | thrombocythemia | | | testing. |
| | myelofibrosis with | | | |
| | CALR and ASXL1 | | | |
| | mutations | | | |
| BMA | B-Cell Lymphoma: CLL with | + | B-Cells: CD19+ CD20−/+ | Previously treated with |
| | 17P deletion | | CD5+ CD10− CD23+/− | ibrutinib and allogeneic |
| | | | CD38− CD200+ with | stem cell transplant with |
| | | | monotypic dim surface lambda | residual disease of 30-40% |
| | | | immunoglobulin light chain | at time of testing |
| | | | expression | (day +82 post transplant). |
| BMA | AML arising from MDS: | − | Myeloid blasts: CD33− CD13− | *Normal B cells +, |
| | recurrence, SRSF2, DNMT3A, | | MPO− CD117+/dim | patient actively on |
| | RUNX1, NOTCH1 mutated. | | CD34+ HLA-DR+. | azacitdine and venetoclax |
| | | | | at time of testing. |
| PBL | B-Cell Lymphocytosis: | + | B-Cells: CD19+ CD20+ CD5+ | No prior therapy, |
| | CLL/SLL with trisomy 12 | | CD10− CD23+ CD38− | on observation. |
| | | | CD200+) with monotypic | |
| | | | moderate surfae | |
| | | | immunoglobulin kappa light | |
| | | | chain expression | |
| PBL | B-Cell lymphoma: Mantle cell | + | 1. 40% B-Cells: CD19+ CD20+ | No prior therapy, |
| | lymphoma. | | CD5dim/− CD10− | on observation. |
| | | | CD23− CD38− CD200dim/− | |
| | | | with monotypic strong | |
| | | | surface kappa immunoglobulin | |
| | | | light chain expression | |
| | | | 2. 9% B-Cells: CD19+ CD20+ | |
| | | | CD5− CD10+ | |
| | | | CD23dim CD38− CD200+ | |
| | | | TdT− with no detectible | |
| | | | surface or cytoplasmic light | |
| | | | chain expression | |
| PBL | B-Cell Lymphoma: Splenic | + | B-Cells: CD19+ CD20+ CD5− | Patient previously treated |
| | Marginal Zone Lymphoma | | CD10− CD23− CD38, | with rituxan monotherapy. |
| | | | CD200− CD25− CD11b | |
| | | | CD11c+/− with monotypic | |
| | | | moderate surface lambda | |
| | | | immunoglobulin light chain | |
| | | | expression | |
| PBL | B-Cell Lymphoma: CLL with | + | B-Cells: CD19+, CD20+, CD5+, | No prior therapy, |
| | trisomy 12 | | CD10−, CD23−/+, | on observation. |
| | | | CD38−, CD200+ with monotypic | |
| | | | moderate surface kappa | |
| | | | immunoglobulin light chain | |
| | | | expression | |
| BMA | AML: In remssion but now has | − | Myeloid blasts: CD33+ CD13+/− | *Normal B cells + |
| | multilineage displasia with | | MPO− CD117+/− | |
| | increased blasts. | | CD34+ HLA-DR+ CD7− | |
| BMA | AML: therapy- related | − | Myeloid Blasts: CD33+ CD13− | *Normal B cells + |
| | | | MPO− CD117+ | |
| | | | CD34+ HLA-DR+ CD56dim CD2− | |
| | | | CD7− CD4− | |
| PBL | B-ALL: JAK2 fusion | − | Lymphoid Blasts: CD19+ CD20+/− | *Normal B cells +, |
| | | | CD5− CD10+ CD45 dim CD10+ | no prior therapy, |
| | | | CD34+ CD33− TdT+ CD38+ | newly diagnosed |
| | | | without surface light chain expression | |
| BMA | APL in remission: t(15; 17) | − | Hematogones: CD19+ CD10+ | *Normal B cells +, |
| | confirmed, also WT1 and | | CD20variable+ | sample following |
| | FLT3-ITD | | | ATRA/Arsenic therapy, |
| | | | | PML/RARA |
| | | | | undetectable by PCR |
| BMA | B-Cell Lymphoma: HCL, | + | B-Cells: CD20+ CD19+ CD5− | No prior therapy. |
| | BRAF V600E and SF3B1 | | CD23− clonal | |
| | mutated | | population with co-expression | |
| | | | of CD25 (dim), CD103 and CD11c | |
| L AXILLARY LN | B-Cell Lymphoma: MCL | + | B-Cells: CD19+ CD20+ CD5+ | No prior therapy. |
| | | | CD10− CD23− CD38+ | |
| | | | CD200− with monotypic strong | |
| | | | surface kappa immunoglobulin | |
| | | | light chain expression | |
| AXILLARY LN | B-Cell Lymphoma: MZL | + | B cells: CD19+ CD20+ | Previously treated |
| | | | CD5−/+ CD10− CD23− | with R-bendamustine |
| | | | CD38− CD200+ with monotypic | and R-CHOP. |
| | | | marked excess of surface | |
| | | | lambda immunoglobulin | |
| | | | light chain compared to kappa. | |
| L NECK LN FNA | B-cell lymphoma with | + | B cells: CD19+ CD20+ CD5− | |
| | plasmacytic | | CD10− CD23−/+ CD38− | |
| | differentiation | | CD200 dim with monotypic moderate | |
| | (IgM kappa+). | | surface kappa immunoglobulin | |
| | | | light chain expression | |
| BMA | AML: recurrence, TET2, | − | Myeloid blasts: CD33+ | *Normal B cells + |
| | STAG2, ASXL1 and CEBPa | | CD13+/− MPO+ CD117+ | |
| | mutated | | CD34+ HLA-DR+ CD56dim CD7+ | |
| PBL | B-Cell Lymphoma: MCL | + | B cells: CD19+ CD20+ CD5+ | Patient previously |
| | | | CD10− CD23− CD38− | treated with R-CVP. |
| | | | CD200−) with monotypic moderate | |
| | | | to strong surface | |
| | | | cytoplasmic kappa immunoglobulin | |
| | | | light chain expression. | |
| | | | CD5+ but with associated t(11; 14). | |
| PBL | B-Cell Lymphoma: CLL | + | B-Cells: CD19+ CD20− CD5+ | No prior therapy, |
| | | | CD10− CD23+ CD38− | on observation. |
| | | | CD200+ with monotypic dim | |
| | | | lambda immunoglobulin | |
| | | | light chain expression | |
| L PELVIC MASS | B-Cell Lymphoma: DLBCL | + | B cells: CD19+ CD20+ CD5− | *Normal B cells + |
| BX | | | CD10− CD23− CD38− | |
| | | | CD200− CD103− CD25− | |
| | | | CD11c− with monotypic strong | |
| | | | surface lambda immunoglobulin | |
| | | | light chain expression. | |
| PBL | Normal | + | B-Cells: CD19+ CD20+ | *Normal B cells + |
| PBL | Normal | + | B-Cells: CD19+ CD20+ | *Normal B cells + |
| PBL | Normal | + | B-Cells: CD19+ CD20+ | *Normal B cells + |
| PBL | Normal | + | B-Cells: CD19+ CD20+ | *Normal B cells + |
| PBL | Normal | + | B-Cells: CD19+ CD20+ | *Normal B cells + |
| PBL | Normal | + | B-Cells: CD19+ CD20+ | *Normal B cells + |
| PBL | Normal | + | B-Cells: CD19+ CD20+ | *Normal B cells + |
| PBL | Normal | + | B-Cells: CD19+ CD20+ | *Normal B cells + |
| BMA | AML: Normal cytogenetics, | − | Myeloid Blasts: CD33+ CD13+ | *Normal B cells +, |
| | no associated mutations. | | MPO−/dim CD117+ | no prior therapy. |
| | | | CD34+ HLA-DR+ | |

In this data set, there were no samples from patients with T cell malignancies. Thus, the expression of CD37 was assessed in primary peripheral T cell lymphomas by performing immunohistochemical staining on a tissue microarray containing triplicate cores of 67 PTCL samples from 9 different subtypes. Overall, positive staining in at least a subset of cells was seen in patient samples from each subtype, including 15 of 16 AILTs, 1 of 1 ALK+ anaplastic large cell lymphomas (ALCLs), 6 of 13 ALK-negative ALCLs, 2 of 6 adult T-cell leukemias, 18 of 23 PTCL, not otherwise specified, 1 of 1 enteropathy-associated T-cell lymphoma, 4 of 4 extranodal NK/T-cell lymphoma, nasal type, 1 of 1 T-cell prolymphocytic leukemia, and 1 of 2 hepatosplenic gamma/delta T cell lymphomas. Representative strong staining for CD37 in a PTCL (ALCL) sample is shown in FIG. 2C.

### Example 14. Generation of Anti-CD37 CAR T Cells

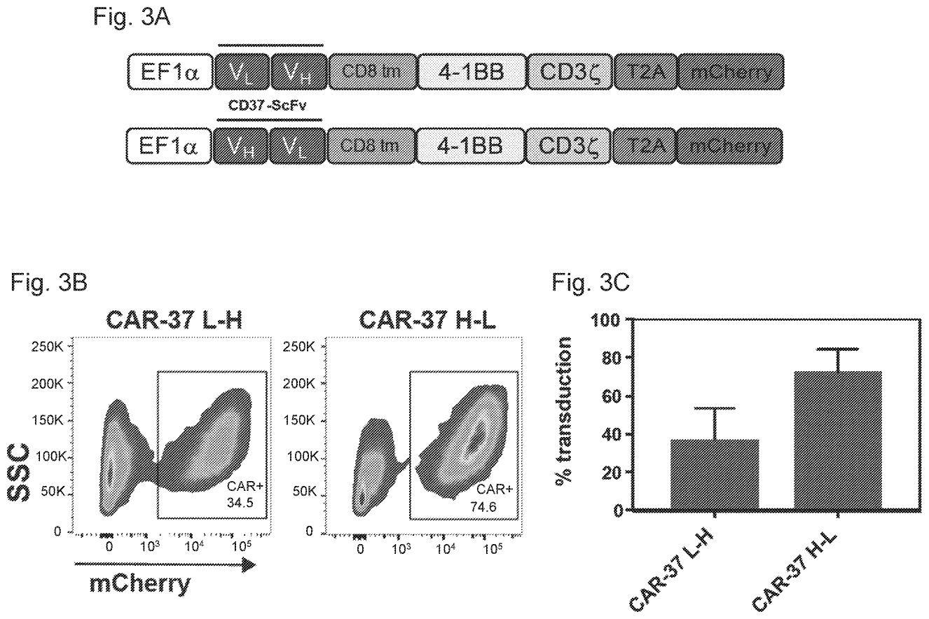

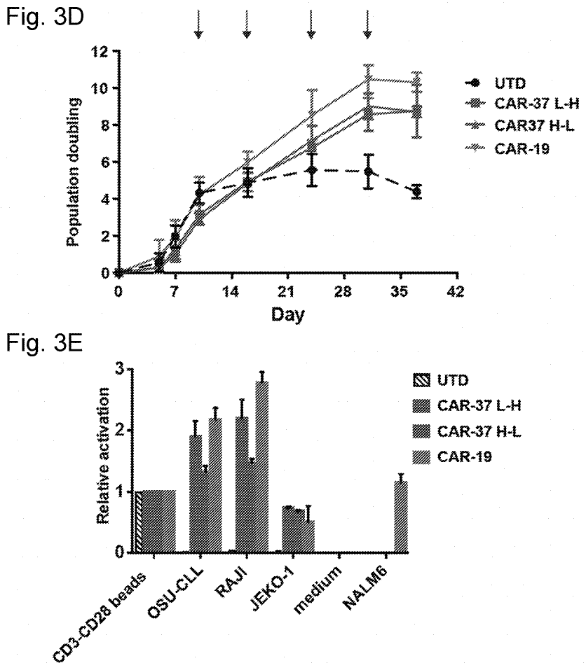

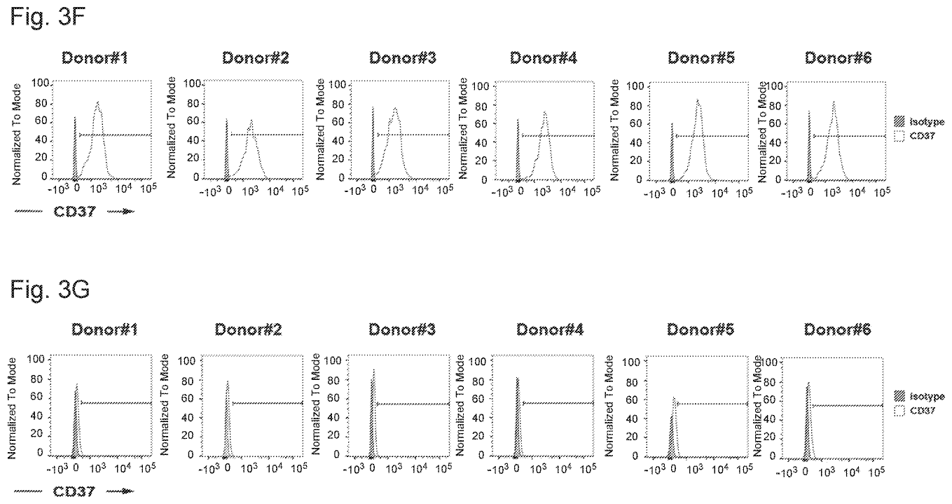

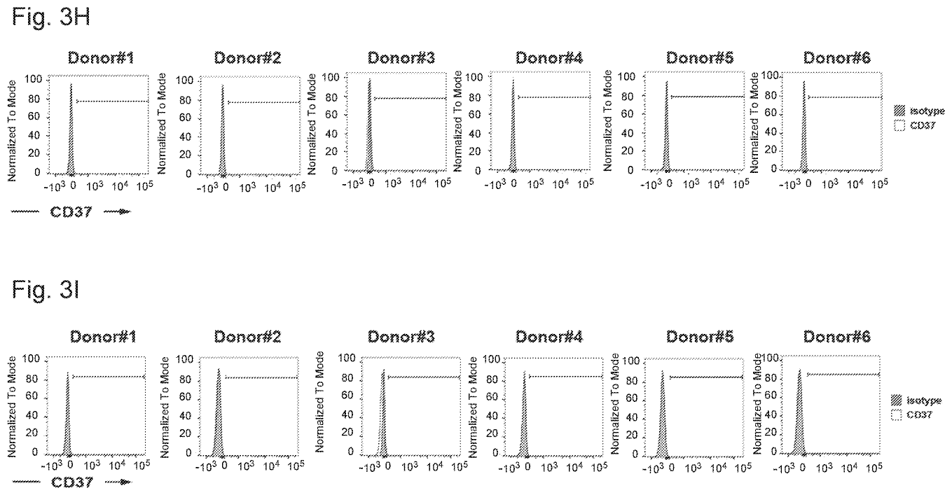

Two anti-CD37 chimeric antigen receptors were designed consisting of anti-CD37 scFv and CD8 transmembrane domain in tandem with 4-1 BB intracellular signaling domain and CD3 (FIG. 3A). The scFvs were synthesized in both orientations of the variable heavy and light chains, generating CAR-37 L-H and CAR-37 H-L. To facilitate evaluation of transduction efficiency, the mCherry fluorescent reporter gene was incorporated following a 2A ribosomal skip sequence at the C-terminal of the CAR sequence. High efficiency gene transfer into primary activated human T cells was obtained with both constructs using 3rd generation self-inactivating lentiviral vectors (FIGS. 3B and 3C). CAR-37 T cells displayed expansion after initial priming with anti-CD3/CD28 beads over the first 10 days, comparable to anti-CD19 CAR T cells (CAR-19). As a comparator, CAR-19 T cells were generated based on the same backbone, with the CD8 transmembrane domain and 4-1 BB and CD3 intracellular signaling domains. It was found that CAR-37 T cells could undergo long-term expansion through repetitive antigen stimulations with irradiated K562 cells transduced to express CD37 and CD19 (FIG. 3D). Next, the activation of the CARs using Jurkat reporter (NFAT-Luciferase) T cells was tested. After transducing the Jurkat reporter cells with the different CAR constructs, the cells were co-cultured them with a variety of stimuli, including anti-CD3/CD28 beads, B cell lymphoma tumor cells, Nalm6 leukemia cells, or media as a negative control. Measurements of the luminescence demonstrated specific T cell activation and NFAT-mediated luminescence in response to antigen stimulation (FIG. 3E). In this assay, anti-CD37 CARs in the L-H orientation appeared to initiate activation more robustly than anti-CD37 CARs in the H-L orientation, and at similar levels as anti-CD19 CARs in response to CD19-expressing tumors. However, there is no known threshold or optimal amount of NFAT translocation that corresponds to an optimal CAR construct. These data indicate that anti-CD37 CARs mediate T cell activation signals in response to specific antigen stimulation and can undergo long-term growth in response to antigen stimulation.

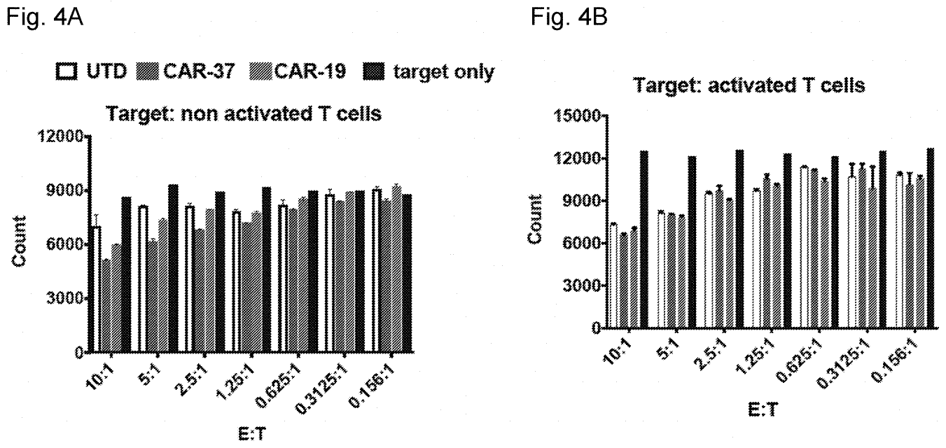

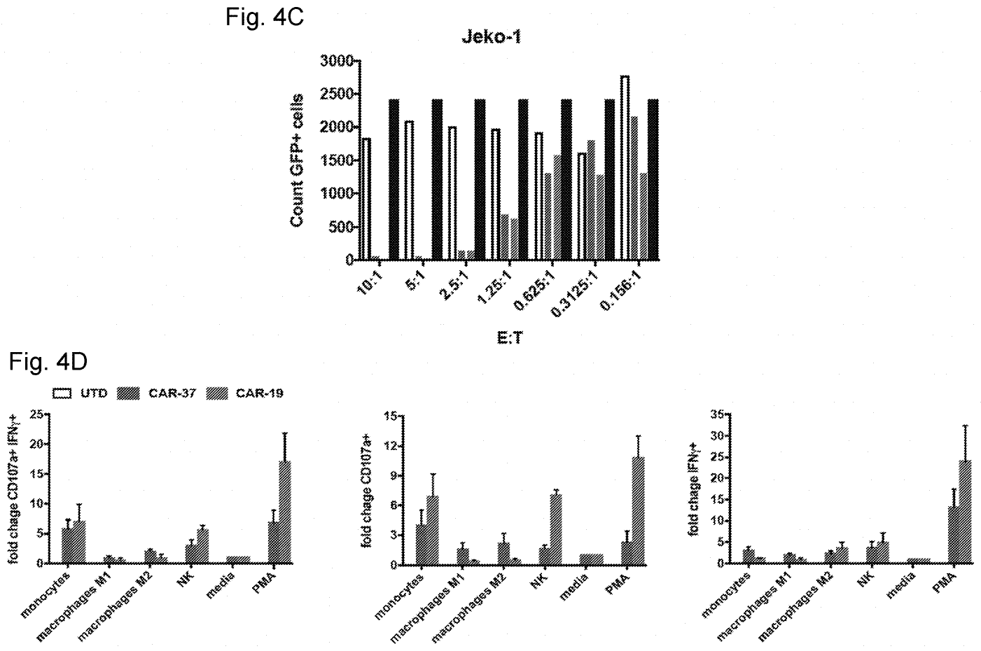

Because CD37 has been reportedly expressed on T cells and other hematopoietic mononuclear cells, the expression of CD37 on whole blood immune cells from healthy donors was interrogated (FIGS. 3F-3I). High expression of CD37 on B cells was observed, with minimal expression on monocytes, but not NK cells or T cells. However, many T cell markers change with activation, and so the possibility of fratricide was tested by co-culturing CAR-37 T cells with activated or non-activated CFSE-labeled T cells from the same donors. After 24 hours, target T cell counts were analyzed. No significant difference was detected in the counts of labeled resting T cells (FIG. 4A) or labeled activated T cells (FIG. 4B) that had been co-cultured with CAR-37 T cells compared to those cultured with CAR-19 T cells, despite expected cytotoxicity against Jeko-1 target cells in the same experiment (FIG. 4C). Next, degranulation and IFNγ production of CAR-37 T cells against monocytes, NK cells, and in vitro differentiated M1 or M2 macrophages was tested (FIG. 4D). No significant difference in degranulation or IFNγ production between CAR-37 and CAR-19 T cells was observed, indicating no evidence of immune-cell toxicity induced by CAR-37 T cells.

### Example 15. CAR-37 T Cells Exhibit Robust Effector Functions in Response to CD37 Positive Tumor Cells In Vitro

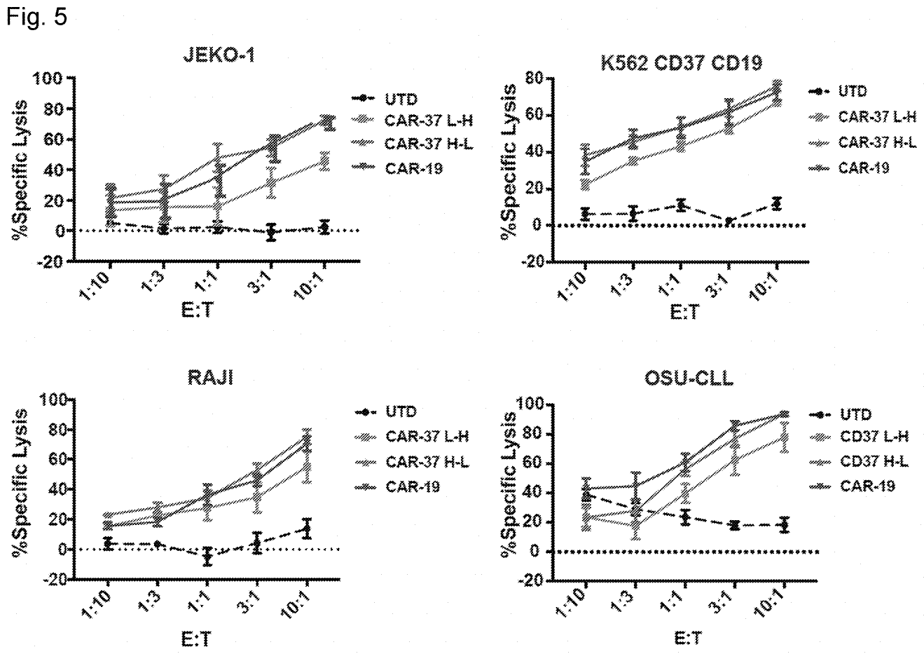

To define the anti-tumor activity of CAR-37 T cells, cytotoxicity assays were performed against a panel of lymphoma cell lines. CAR-37 T cells were co-cultured with Jeko-1, OSU-CLL, Raji or K562-CD37-CD19 cells at various effector to target ratios for 16 hours (FIG. 5). All CAR-37 T cell effector were able to lyse target cells, but in contrast to what was observed in the Jurkat activation assay, the heavy-light chain configuration was more favorable than the light-heavy configuration for anti-CD37 CAR T cells. This difference was evident and consistent with all the tumor lines tested. Notably, T cells transduced with CAR-37 H-L or CAR-19 demonstrated equivalent cytolytic activity against these target tumor cells, all of which express both antigens.

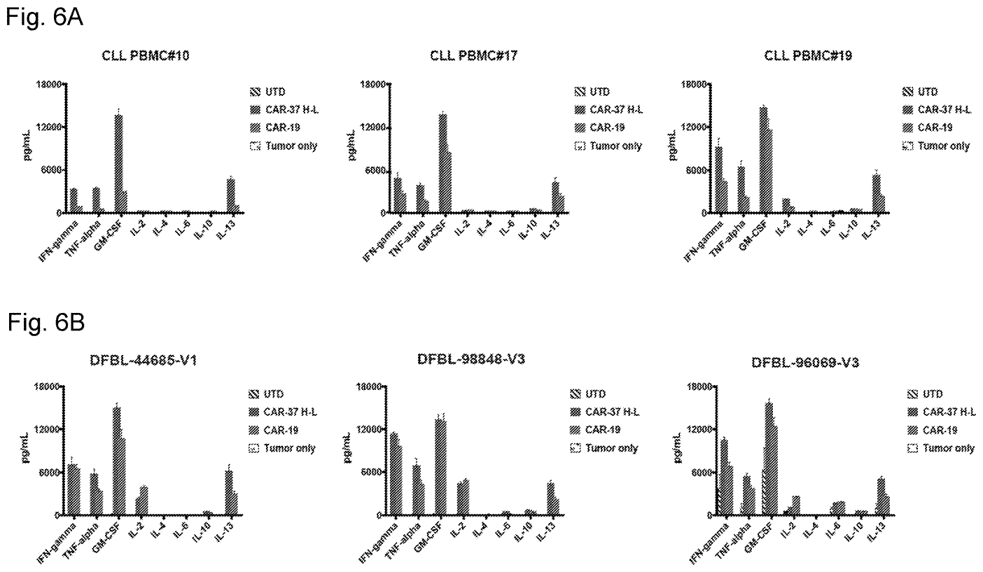

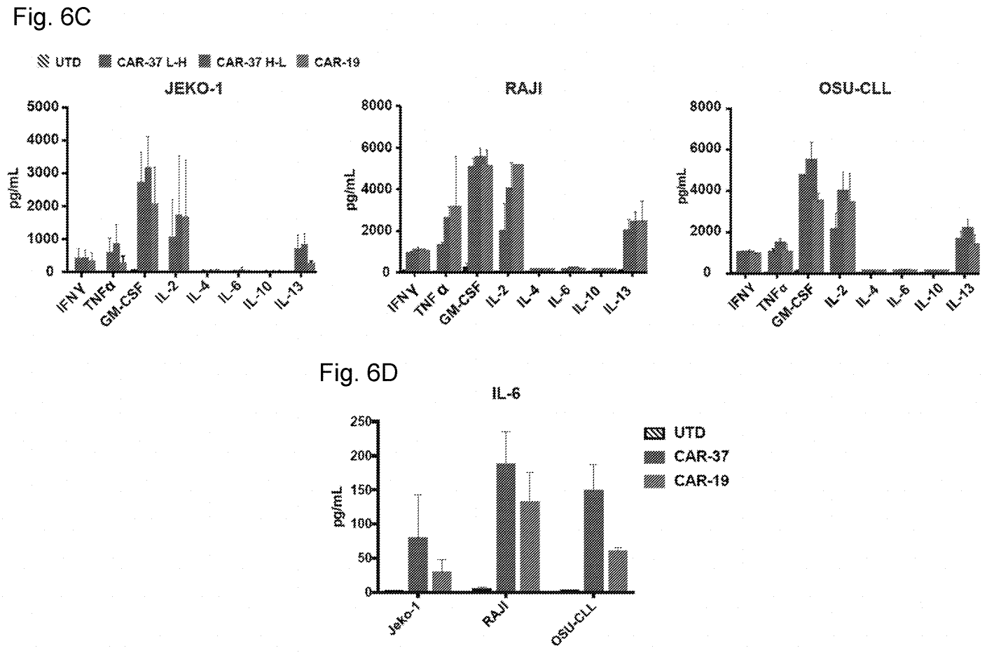

Next, cytokine production in response to antigen stimulation was analyzed. The different patterns of cytokines produced by different CAR constructs after stimulation with target cells were compared. CAR-37 T cells demonstrated antigen-specific production of the Th1-type cytokines TNF-α, IFN-γ, IL-2, and GM-CSF after in vitro stimulation with tumor cell lines (FIGS. 6C and 6D), primary CLL (FIG. 6A) and MCL patient-derived xenograft (PDX) samples (FIG. 6B). Consistent with the cytotoxicity assays, these experiments demonstrate improved antigen-specific effector function of CAR-37 H-L compared to CAR-37 L-H.

### Example 16. CAR-37 T Cells Eradicate MCL Tumor In Vivo

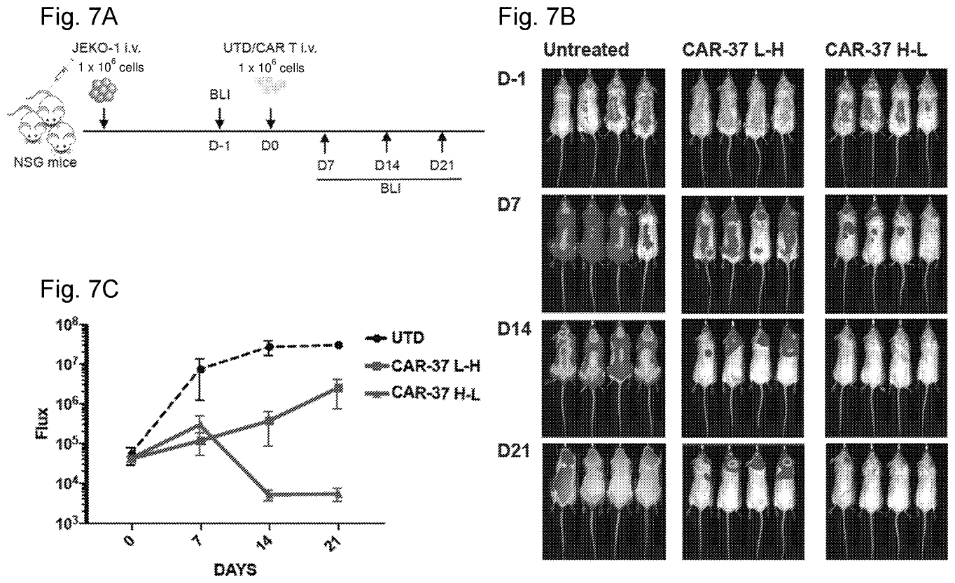

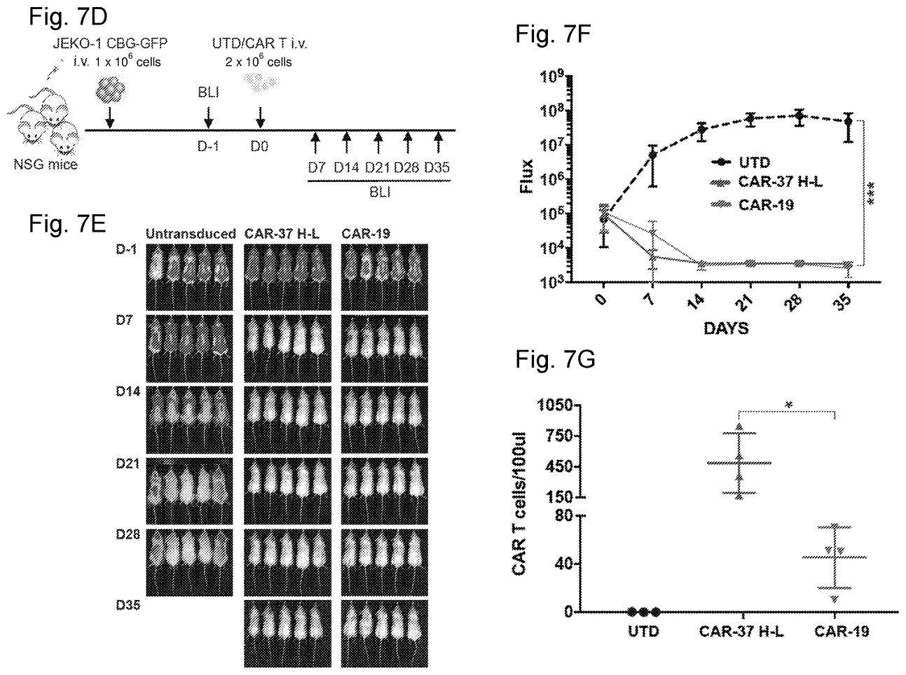

Because the in vitro assays indicated that CAR-37 H-L was possibly, but not definitively, superior to CAR-37 L-H, these two formats were compared in a xenogeneic model of MCL. NSG mice were injected intravenously with luciferase-expressing Jeko-1 (CBG-GFP+) cells. Seven days later, disease burden was assessed by bioluminescence imaging (BLI) in all mice, and CAR-37 or untransduced (UTD) T cells were administered by tail vein injection. By 14 days, there was partial disease control in CAR-37 L-H treated animals but complete disease eradication in CAR-37 H-L treated mice (FIGS. 7A-7C), thus confirming the superior antigen-induced effector function of CAR-37 H-L over CAR-37 L-H, which was then selected for further experiments and for the direct comparison with CAR-19 T cells in vivo. CAR T cells were injected into NSG mice seven days after intravenous injection of the MCL cell line Jeko-1 (CBG-GFP+) as shown (FIG. 7D). Serial imaging of luminescence to assess tumor burden indicated rapid and complete elimination of tumor by day 14 for both CAR-37 and CAR-19 T cells, with dramatic reductions in tumor volume by day 7 (FIGS. 7E and 7F). CAR T-cell persistence was confirmed in the peripheral blood by flow cytometry (FIG. 7G), with greater persistence of CAR-37 T cells at day 7 (p<0.05).

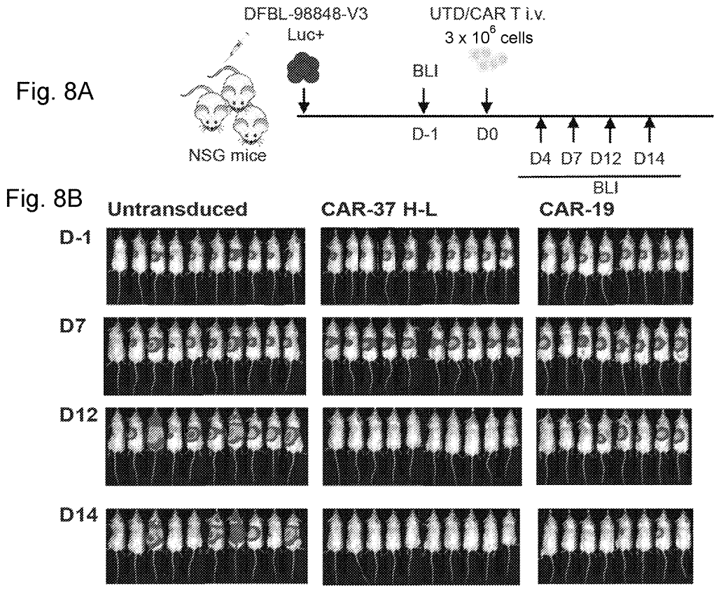

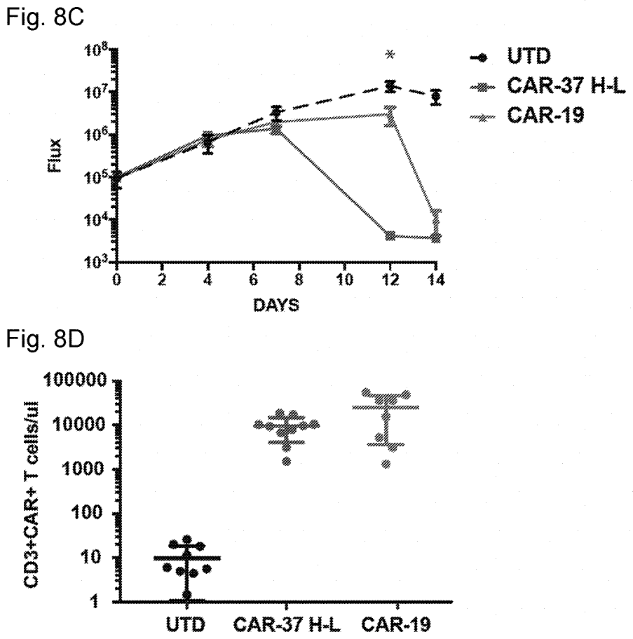

Although tumor cell lines are quite useful in the assessment of efficacy of CAR T cells, they often do not fully represent the heterogeneity and biology of primary patient tumors. In contrast, PDX models, where tumor cells are derived directly from patients and cultured for only 2-3 passages, are thought to resemble the clinical setting more closely. The efficacy of CAR-37 (H-L configuration) in PDX models of MCL was assessed. Ten NSG mice were injected with luciferase-expressing MCL-PDX cells. After confirming disease engraftment and tumor burden similar to the Jeko-1 model by BLI, CAR-37, CAR-19 or untransduced T cells were injected (FIG. 8A). It was observed that CAR-37 T cells were able to clear the tumor in only 12 days, notably faster than CAR-19 (p<0.05 at the day 12 time point) (FIGS. 8B and 8C). Flow cytometry assessment of peripheral blood collected at day 14 confirmed the persistence of CAR T cells in the blood (FIG. 8D). Collectively, these results indicate that CAR-37 T cells mediate significant antitumor effects against B-cell NHL in vivo, in both tumor-line and PDX-models of MCL.

### Example 17. Targeting CD37 on T Cell Lymphoma

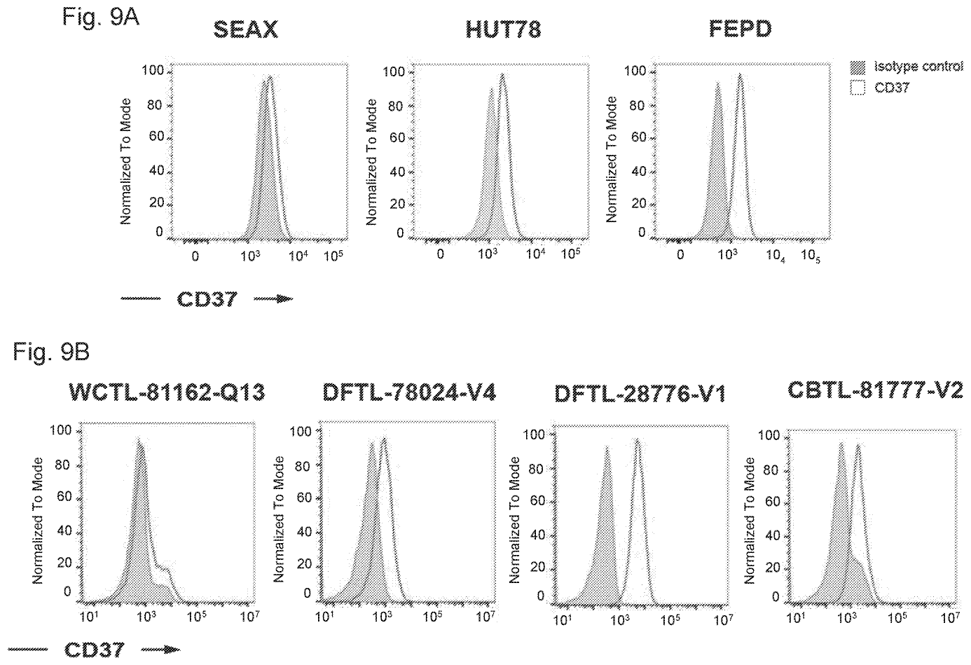

The surface expression of CD37 in PTCL lines and PDX samples of PTCL was analyzed by flow cytometry. Three cell lines were identified (Hut78, Fedp and Seax) and five PDX samples that expressed CD37 on the cell surface at varying levels (FIGS. 9A and 9B, Table 3).

TABLE 3

| PTCL PDX | CLASSIFICATION | CD37 |
|---|---|---|
| WCTL-81162-Q13 | Anaplastic large cell lymphoma, ALK positive | − |
| DFTL-78024-V4 | Angioimmunoblastic T-cell lymphoma | + |
| DFTL-28776-V1 | T-cell prolymphocytic leukemia | + |
| DFTL-22685-V4 | Primary cutaneous CD30+ T-cell lymphoproliferative disorder | + |
| DFTL-47880-V1 | Angioimmunoblastic T-cell lymphoma | + |
| CBTL-81777-V2 | Hepatosplenic T-cell lymphoma | + |
| DFTL-85005-V4 | Extranodal NK/T-cell lymphoma | − |

The activation of CAR-37 T cells following co-culture with these PTCL samples was tested. The CD69 activation marker was analyzed by flow cytometry on gated CAR+ T cells; robust upregulation of CD69 after 6 hours of co-culture with PTCL target cells was observed, indicating CAR-37 T cell activation (FIG. 10A). Interestingly, the degree of CD69 upregulation was independent of the level of expression of the CD37 antigen on the target cells. Next, the degranulation of CAR-37 T cells was tested by measuring expression of CD107a in CAR-37 cells after incubation with PTCL target cells (FIG. 10B). Consistent with the CD69 assays, it was observed that CAR-37 T cells degranulated in response to PTCL cells, indicating activation of CAR-37 T cells in response to PTCL. Finally, it was observed that CAR-37 T cells effectively lysed the CD37 positive PTCL cell lines Hut78 and Fepd, as determined by cytotoxicity assays performed at varying E:T ratios (FIGS. 10C and 10D). Taken together, these experiments demonstrate that CAR-37 T cells are activated against and lyse T-cell lymphoma cells.

### Example 18. Bispecific CAR T Cells Against CD19 and CD37

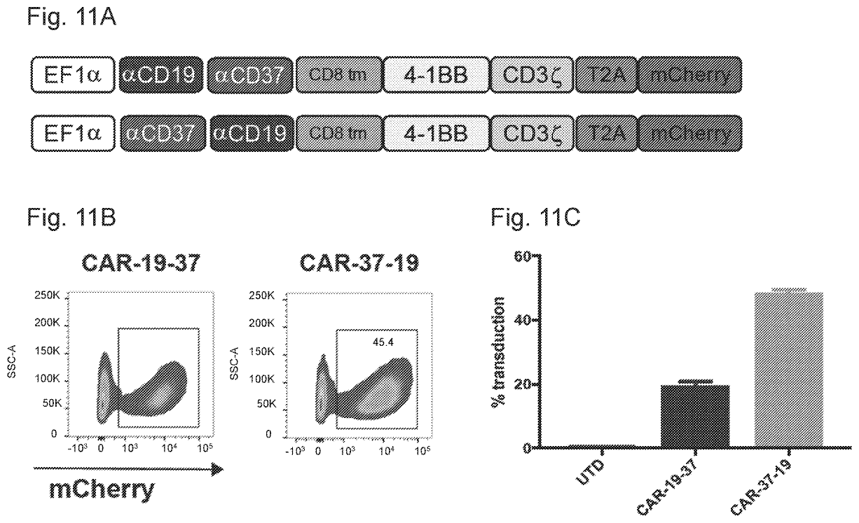

A possible strategy to avoid antigen escape is to generate T cells capable of recognizing multiple antigens (Ruella et al., J. Clin. Invest. 126:3814-26, 2016; Zah et al., Cancer Immunology Research. 4:498-508, 2016). A dual targeting of CD19 and CD37 has been previously investigated with a dual-ligand immunoliposomes approach which resulted effective on B-CLL cells (Yu et al., Biomaterials. 34:6185-6193, 2013). A bispecific CAR was designed that would efficiently trigger T-cell activation when CD19 or CD37 are present on the target cell alone or in combination. Two constructs were generated and tested carrying anti-CD19 and anti-CD37 scFvs connected in tandem in different orders in a second generation 4-1 BB-CD3ζ vector (FIG. 11A). It was observed that the transduction efficiency was low for CAR19-37 T cells compared to CAR37-19, even if the same MOI was used (FIGS. 11B and 11C).

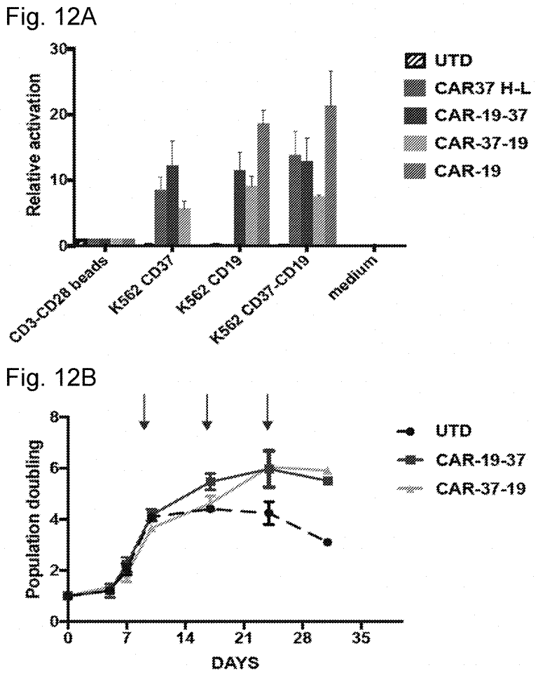

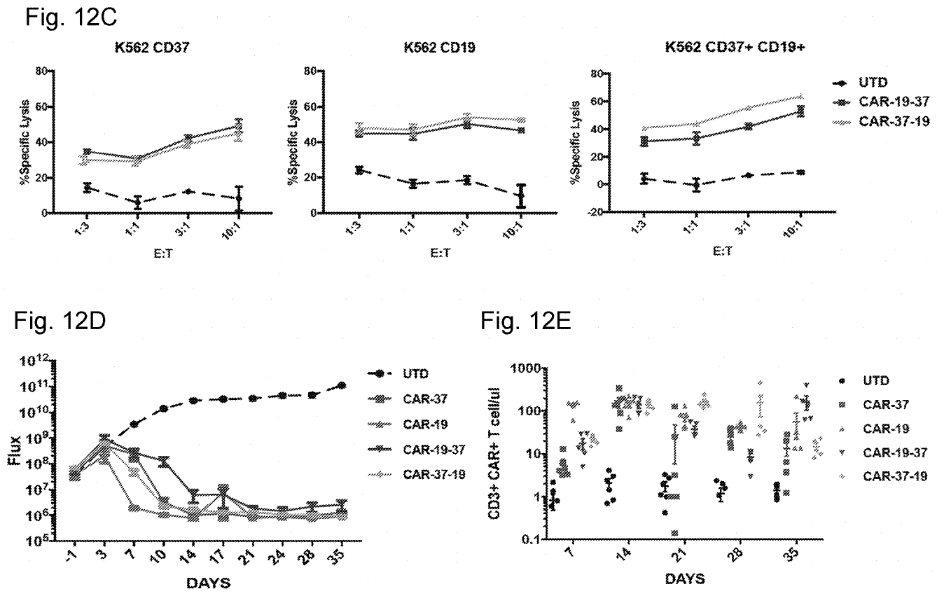

To determine whether bispecific CAR T cells could be activated in response to either antigen, CAR-19, CAR-37, bispecific CAR19-37, and bispecific CAR37-19 Jurkat NFAT-Luc reporter cell lines were generated and analyzed activation after overnight co-culture with target cells. Bispecific CAR T cells showed high NFAT activation when either a single or both antigens were present on the surface of the target cells, and there was no discernible difference in activation signal in bispecific CAR T cells when they were activated by CD19 alone or CD37 alone (FIG. 12A). Bispecific CAR T cells displayed comparable expansion after initial priming with anti-CD3/CD28 beads over the first 10 days (FIG. 12B). Similarly, it was found that bispecific CAR T cells could undergo long-term expansion through repetitive antigen stimulations with K562 expressing CD19 and CD37 (FIG. 12B). In a cytotoxicity assay with primary human CAR T cells, it was observed that bispecific CAR T cells responded to either a single target or both targets, and there was no discernible difference in cytotoxicity to CD19 or CD37 (FIG. 12C). Lastly, the bispecific constructs were tested in vivo against Jeko-1 tumor cells, compared against conventional CAR-37 and CAR-19 T cells. By day 14 after treatment, there was complete disease eradication in all groups except CAR-19-37, which showed partial disease control (FIG. 12D). This discrepancy may be related to structural differences between the two antigens and the order of the scFvs in the CAR constructs. CAR T-cell persistence in the peripheral blood was confirmed by flow cytometry through day 35 of treatment (FIG. 12E). There was no discernible difference between CAR-37, CAR-19, and the optimal tumor-clearing bispecific CAR37-19 at all of the time points examined. Interestingly, the sub-optimal CAR 19-37 had greater persistence at day 35, which may reflect persistent antigen stimulation by tumor. Taken together, these data indicate that bispecific CAR T cells caused specific target cytolysis of cells expressing one or both antigens on the surface and that they are as effective as either mono-specific CAR alone against MCL tumor in vivo.

### Example 19. CAR Immunotherapy with NK Cells

CD37 expression has been reported in PTCL (Pereira et al., Mol. Cancer. Ther. 14(7):1650-60, 2015) and many B-NHLs and thus is a promising target for CAR T cell immunotherapy. However, activated T cells can express CD37, yet natural killer (NK) cells do not. NK cells thus are an attractive cytotoxicity cell for CAR T cell therapy including CD19/CD37-CAR or CD37-CAR. NK cells mediate anti-tumor effects without the risk of GvHD and are short-lived relative to T cells. This also makes NK cells, with or without CAR-37, promising sources for allogeneic cellular therapy products. Previous pre-clinical studies have redirected CAR modified primary human NK cells against different antigens including CD19, CD20, and HER2, and anti-CD19 CAR-modified, donor-derived, and haploidentical NK cells have entered clinical trials for B cell ALL (NCT00995137, NCT01974479). T cell self-targeting can potentially be mitigated by using NK cells instead, as well as using NKs as an allogeneic source.

### Example 20. Sequences

scFv sequences

Anti-CD37 scFv VH-VL (SEQ ID NO: 4) comprising a VH (amino acids 1-116 (SEQ ID NO: 1)), a linker
region (amino acids 117-136 (SEQ ID NO: 3)), and a VL (amino acids 137-244 (SEQ ID NO: 2)).
AVQLVQSGAEVKKPGSSVKVSCKASGYSFTGYNMNWVRQAPGQGLEWMGNIDPYYGGTTYN
RKFKGRVTLTVDKSSSTAYMELSSLRSEDTAVYYCARSVGPMDYWGQGTLVTVSSGGGGSGG
GGSGGGGSGGGGSDIQMTQSPSSLSASVGDRVTITCRTSENVYSYLAWYQQKPGKAPKLLVSS
AKTLAEGVPSRFSGSGSGTDFTLTISSLQPEDFATYFCQHHSDNPWTFGQGTKVEIKR (SEQ ID NO: 4)
VH (SEQ ID NO: 1 (amino acids 1-116 of SEQ ID NO: 4))
AVQLVQSGAEVKKPGSSVKVSCKASGYSFTGYNMNWVRQAPGQGLEWMGNIDPYYGGTTYN
RKFKGRVTLTVDKSSSTAYMELSSLRSEDTAVYYCARSVGPMDYWGQGTLVTVSS (SEQ ID NO: 1)
Linker region (SEQ ID NO: 3 (amino acids 117-136 of SEQ ID NO: 4))
GGGGSGGGGSGGGGSGGGGS (SEQ ID NO: 3)
VL (SEQ ID NO: 2 (amino acids 137-244 SEQ ID NO: 4))
DIQMTQSPSSLSASVGDRVTITCRTSENVYSYLAWYQQKPGKAPKLLVSSAKTLAEGVPSRFSG
SGSGTDFTLTISSLQPEDFATYFCQHHSDNPWTFGQGTKVEIKR (SEQ ID NO: 2)
Anti-CD37 scFv VL-VH (SEQ ID NO: 5) comprising a VL (amino acids 1-108 (SEQ ID NO: 2)), a linker
region (amino acids 109-128 (SEQ ID NO: 3)), and a VH (amino acids 129-244 (SEQ ID NO: 1).
DIQMTQSPSSLSASVGDRVTITCRTSENVYSYLAWYQQKPGKAPKLLVSSAKTLAEGVPSRFSG
SGSGTDFTLTISSLQPEDFATYFCQHHSDNPWTFGQGTKVEIKRGGGGSGGGGSGGGGSGGG
GSAVQLVQSGAEVKKPGSSVKVSCKASGYSFTGYNMNWVRQAPGQGLEWMGNIDPYYGGTT
YNRKFKGRVTLTVDKSSSTAYMELSSLRSEDTAVYYCARSVGPMDYWGQGTLVTVSS (SEQ ID NO: 5)
VL (SEQ ID NO: 2 (amino acids 1-108 of SEQ ID NO: 5))
DIQMTQSPSSLSASVGDRVTITCRTSENVYSYLAWYQQKPGKAPKLLVSSAKTLAEGVPSRFSG
SGSGTDFTLTISSLQPEDFATYFCQHHSDNPWTFGQGTKVEIKR (SEQ ID NO: 2)
Linker region (SEQ ID NO: 3 (amino acids 109-128 of SEQ ID NO: 5))
GGGGSGGGGSGGGGSGGGGS (SEQ ID NO: 3)
VH (SEQ ID NO: 1 (amino acids 129-244 SEQ ID NO: 5))
AVQLVQSGAEVKKPGSSVKVSCKASGYSFTGYNMNWVRQAPGQGLEWMGNIDPYYGGTTYN
RKFKGRVTLTVDKSSSTAYMELSSLRSEDTAVYYCARSVGPMDYWGQGTLVTVSS (SEQ ID NO: 1)
Anti-CD19 scFv (SEQ ID NO: 14) comprising a VL (amino acids 1-107 (SEQ ID NO: 13)), a linker region
(amino acids 108-128 (SEQ ID NO: 3)), and a VH (amino acids 129-247 (SEQ ID NO: 12)).
EIVMTQSPATLSLSPGERATLSCRASQDISKYLNWYQQKPGQAPRLLIYHTSRLHSGIPARFSGS
GSGTDYTLTISSLQPEDFAVYFCQQGNTLPYTFGQGTKLEIKGGGGSGGGGSGGGGSGGGGS
QVQLQESGPGLVKPSETLSLTCTVSGVSLPDYGVSWIRQPPGKGLEWIGVIWGSETTYYQSSLK
SRVTISKDNSKNQVSLKLSSVTAADTAVYYCAKHYYYGGSYAMDYWGQGTLVTVSS (SEQ ID NO: 14)
VL (SEQ ID NO: 13 (amino acids 1-107 of SEQ ID NO: 14))
EIVMTQSPATLSLSPGERATLSCRASQDISKYLNWYQQKPGQAPRLLIYHTSRLHSGIPARFSGS
GSGTDYTLTISSLQPEDFAVYFCQQGNTLPYTFGQGTKLEIK (SEQ ID NO: 13)
Linker region (SEQ ID NO: 3 (amino acids 108-128 of SEQ ID NO: 14)
GGGGSGGGGSGGGGSGGGGS (SEQ ID NO: 3)
VH (SEQ ID NO: 12 (amino acids 129-247 of SEQ ID NO: 14))
QVQLQESGPGLVKPSETLSLTCTVSGVSLPDYGVSWIRQPPGKGLEWIGVIWGSETTYYQSSLK
SRVTISKDNSKNQVSLKLSSVTAADTAVYYCAKHYYYGGSYAMDYWGQGTLVTVSS (SEQ ID NO: 12)
CAR sequences
pMGH8 (CAR-37 L-H) - CD8 signal/anti-CD37 L-H/CD8 hinge + TM/4-1BB/CD3ζ (SEQ ID NO: 7)
comprising CD8 signal sequence (amino acids 1-21 (SEQ ID NO: 8)); anti-CD37 L-H (amino acids 22-265
(SEQ ID NO: 5)); CD8 hinge and TM domain (amino acids 266-334 (SEQ ID NO: 9)); 4-1BB (amino acids
335-376 (SEQ ID NO: 10)); and CD3ζ (amino acids 377-488 (SEQ ID NO: 11)).
MALPVTALLLPLALLLHAARPDIQMTQSPSSLSASVGDRVTITCRTSENVYSYLAWYQQKPGKAP
KLLVSSAKTLAEGVPSRFSGSGSGTDFTLTISSLQPEDFATYFCQHHSDNPWTFGQGTKVEIKR
GGGGSGGGGSGGGGSGGGGSAVQLVQSGAEVKKPGSSVKVSCKASGYSFTGYNMNWVRQA
PGQGLEWMGNIDPYYGGTTYNRKFKGRVTLTVDKSSSTAYMELSSLRSEDTAVYYCARSVGPM
DYWGQGTLVTVSSTTTPAPRPPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR (SEQ ID NO: 7)
CD8 signal sequence (SEQ ID NO: 8 (amino acids 1-21 of SEQ ID NO: 7))
MALPVTALLLPLALLLHAARP (SEQ ID NO: 8)
Anti-CD37 L-H (SEQ ID NO: 5 (amino acids 22-265 of SEQ ID NO: 7))
DIQMTQSPSSLSASVGDRVTITCRTSENVYSYLAWYQQKPGKAPKLLVSSAKTLAEGVPSRFSG
SGSGTDFTLTISSLQPEDFATYFCQHHSDNPWTFGQGTKVEIKRGGGGSGGGGSGGGGSGGG
GSAVQLVQSGAEVKKPGSSVKVSCKASGYSFTGYNMNWVRQAPGQGLEWMGNIDPYYGGTT
YNRKFKGRVTLTVDKSSSTAYMELSSLRSEDTAVYYCARSVGPMDYWGQGTLVTVSS (SEQ ID NO: 5)
CD8 hinge and TM domains SEQ ID NO: 9 (amino acids 266-334 of SEQ ID NO: 7)
TTTPAPRPPTPAPTIASQPLSLRPEACRPAAGGAVHTRGLDFACDIYIWAPLAGTCGVLLLSLVITL
YC (SEQ ID NO: 9)
4-1BB (SEQ ID NO: 10 (amino acids 335-376 of SEQ ID NO: 7))
KRGRKKLLYIFKQPFMRPVQTTQEEDGCSCRFPEEEEGGCEL (SEQ ID NO: 10)
CD3ζ (SEQ ID NO: 11 (amino acids 377-488 of SEQ ID NO: 7))
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR (SEQ ID NO: 11)
pMGH8 (CAR-37 H-L) - CD8 signal/anti-CD37 H-L/CD8 hinge + TM/4-1BB/CD3ζ (SEQ ID NO: 6)
comprising CD8 signal sequence (amino acids 1-21 (SEQ ID NO: 8)); anti-CD37 H-L (amino acids 22-265
(SEQ ID NO: 4)); CD8 hinge and TM domain (amino acids 266-334 (SEQ ID NO: 9)); 4-1BB (amino acids
335-376 (SEQ ID NO: 10)); and CD3ζ (amino acids 377-488 (SEQ ID NO: 11)).
MALPVTALLLPLALLLHAARPAVQLVQSGAEVKKPGSSVKVSCKASGYSFTGYNMNWVRQAPG
QGLEWMGNIDPYYGGTTYNRKFKGRVTLTVDKSSSTAYMELSSLRSEDTAVYYCARSVGPMDY
WGQGTLVTVSSGGGGSGGGGSGGGGSGGGGSDIQMTQSPSSLSASVGDRVTITCRTSENVY
SYLAWYQQKPGKAPKLLVSSAKTLAEGVPSRFSGSGSGTDFTLTISSLQPEDFATYFCQHHSDN
PWTFGQGTKVEIKRTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFS
RSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR (SEQ ID NO: 6)
CD8 signal sequence (SEQ ID NO: 8 (amino acids 1-21 of SEQ ID NO: 6))
MALPVTALLLPLALLLHAARP (SEQ ID NO: 8)
Anti-CD37 H-L (SEQ ID NO: 4 (amino acids 22-265 of SEQ ID NO: 6))
AVQLVQSGAEVKKPGSSVKVSCKASGYSFTGYNMNWVRQAPGQGLEWMGNIDPYYGGTTYN
RKFKGRVTLTVDKSSSTAYMELSSLRSEDTAVYYCARSVGPMDYWGQGTLVTVSSGGGGSGG
GGSGGGGSGGGGSDIQMTQSPSSLSASVGDRVTITCRTSENVYSYLAWYQQKPGKAPKLLVSS
AKTLAEGVPSRFSGSGSGTDFTLTISSLQPEDFATYFCQHHSDNPWTFGQGTKVEIKR (SEQ ID NO: 4)
CD8 hinge and TM domains (SEQ ID NO: 9 (amino acids 266-334 of SEQ ID NO: 6))
TTTPAPRPPTPAPTIASQPLSLRPEACRPAAGGAVHTRGLDFACDIYIWAPLAGTCGVLLLSLVITL
YC (SEQ ID NO: 9)
4-1BB (SEQ ID NO: 10 (amino acids 335-376 of SEQ ID NO: 6))
KRGRKKLLYIFKQPFMRPVQTTQEEDGCSCRFPEEEEGGCEL (SEQ ID NO: 10)
CD3ζ (SEQ ID NO: 11 (amino acids 377-488 of SEQ ID NO: 6))
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR (SEQ ID NO: 11)
pMGH95 (CAR-19-37) - CD8 signal/anti-CD19/anti-CD37 H-L/CD8 hinge + TM/4-1BB/CD3ζ (SEQ
ID NO: 15) comprising CD8 signal sequence (amino acids 1-21 (SEQ ID NO: 8)); anti-CD19 (amino acids
22-268 (SEQ ID NO: 14)); linker (amino acids 269-288 (SEQ ID NO: 3)); anti-CD37 H-L (amino acids
289-532 (SEQ ID NO: 4)); CD8 hinge + TM (amino acids 533-601 (SEQ ID NO: 9)); 4-1BB (amino acids
602-643 (SEQ ID NO: 10)), CD3ζ (amino acids 644-755 (SEQ ID NO: 11)).
MALPVTALLLPLALLLHAARPEIVMTQSPATLSLSPGERATLSCRASQDISKYLNWYQQKPGQAP
RLLIYHTSRLHSGIPARFSGSGSGTDYTLTISSLQPEDFAVYFCQQGNTLPYTFGQGTKLEIKGGG
GSGGGGSGGGGSGGGGSQVQLQESGPGLVKPSETLSLTCTVSGVSLPDYGVSWIRQPPGKGL
EWIGVIWGSETTYYQSSLKSRVTISKDNSKNQVSLKLSSVTAADTAVYYCAKHYYYGGSYAMDY
WGQGTLVTVSSGGGGSGGGGSGGGGSGGGGSAVQLVQSGAEVKKPGSSVKVSCKASGYSF
TGYNMNWVRQAPGQGLEWMGNIDPYYGGTTYNRKFKGRVTLTVDKSSSTAYMELSSLRSEDT
AVYYCARSVGPMDYWGQGTLVTVSSGGGGSGGGGSGGGGSGGGGSDIQMTQSPSSLSASV
GDRVTITCRTSENVYSYLAWYQQKPGKAPKLLVSSAKTLAEGVPSRFSGSGSGTDFTLTISSLQP
EDFATYFCQHHSDNPWTFGQGTKVEIKRTTTPAPRPPTPAPTIASQPLSLRPEACRPAAGGAVH
TRGLDFACDIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR (SEQ ID NO: 15)
CD8 signal sequence (SEQ ID NO: 8 (amino acids 1-21 of SEQ ID NO: 15))
MALPVTALLLPLALLLHAARP (SEQ ID NO: 8)
Anti-CD19 (SEQ ID NO: 14 (amino acids 22-268 of SEQ ID NO: 15)
EIVMTQSPATLSLSPGERATLSCRASQDISKYLNWYQQKPGQAPRLLIYHTSRLHSGIPARFSGS
GSGTDYTLTISSLQPEDFAVYFCQQGNTLPYTFGQGTKLEIKGGGGSGGGGSGGGGSGGGGS
QVQLQESGPGLVKPSETLSLTCTVSGVSLPDYGVSWIRQPPGKGLEWIGVIWGSETTYYQSSLK
SRVTISKDNSKNQVSLKLSSVTAADTAVYYCAKHYYYGGSYAMDYWGQGTLVTVSS (SEQ ID NO: 14)
Linker region (SEQ ID NO: 3 (amino acids 269-288 of SEQ ID NO: 15)
GGGGSGGGGSGGGGSGGGGS (SEQ ID NO: 3)
Anti-CD37 H-L (SEQ ID NO: 4 (amino acids 289-532 of SEQ ID NO: 15))
AVQLVQSGAEVKKPGSSVKVSCKASGYSFTGYNMNWVRQAPGQGLEWMGNIDPYYGGTTYN
RKFKGRVTLTVDKSSSTAYMELSSLRSEDTAVYYCARSVGPMDYWGQGTLVTVSSGGGGSGG
GGSGGGGSGGGGSDIQMTQSPSSLSASVGDRVTITCRTSENVYSYLAWYQQKPGKAPKLLVSS
AKTLAEGVPSRFSGSGSGTDFTLTISSLQPEDFATYFCQHHSDNPWTFGQGTKVEIKR (SEQ ID NO: 4)
CD8 hinge and TM domains (SEQ ID NO: 9 (amino acids 533-601 of SEQ ID NO: 15));
TTTPAPRPPTPAPTIASQPLSLRPEACRPAAGGAVHTRGLDFACDIYIWAPLAGTCGVLLLSLVITL
YC (SEQ ID NO: 9)
4-1BB (SEQ ID NO: 10 (amino acids 602-643 of SEQ ID NO: 15)),
KRGRKKLLYIFKQPFMRPVQTTQEEDGCSCRFPEEEEGGCEL (SEQ ID NO: 10)
CD3ζ (SEQ ID NO: 11 (amino acids 644-755 of SEQ ID NO: 15))
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR (SEQ ID NO: 11)
pMGH96 (CAR-37-19) - CD8 signal/anti-CD37 H-L/anti-CD19/CD8 hinge + TM/4-1BB/CD3ζ (SEQ
ID NO: 16) comprising CD8 signal sequence (amino acids 1-21 (SEQ ID NO: 8)); anti-CD37 H-L (amino
acids 22-265 (SEQ ID NO: 4)); linker (amino acids 266-285 (SEQ ID NO: 3)); anti-CD19 (amino acids
286-532 (SEQ ID NO: 14)); CD8 hinge + TM (amino acids 533-601 (SEQ ID NO: 9)); 4-1BB (amino acids
602-643 (SEQ ID NO: 10)), CD3ζ (amino acids 644-755 (SEQ ID NO: 11)).
MALPVTALLLPLALLLHAARPAVQLVQSGAEVKKPGSSVKVSCKASGYSFTGYNMNWVRQAPG
QGLEWMGNIDPYYGGTTYNRKFKGRVTLTVDKSSSTAYMELSSLRSEDTAVYYCARSVGPMDY
WGQGTLVTVSSGGGGSGGGGSGGGGSGGGGSDIQMTQSPSSLSASVGDRVTITCRTSENVY
SYLAWYQQKPGKAPKLLVSSAKTLAEGVPSRFSGSGSGTDFTLTISSLQPEDFATYFCQHHSDN
PWTFGQGTKVEIKRGGGGSGGGGSGGGGSGGGGSEIVMTQSPATLSLSPGERATLSCRASQD
ISKYLNWYQQKPGQAPRLLIYHTSRLHSGIPARFSGSGSGTDYTLTISSLQPEDFAVYFCQQGNT
LPYTFGQGTKLEIKGGGGSGGGGSGGGGSGGGGSQVQLQESGPGLVKPSETLSLTCTVSGVS
LPDYGVSWIRQPPGKGLEWIGVIWGSETTYYQSSLKSRVTISKDNSKNQVSLKLSSVTAADTAVY
YCAKHYYYGGSYAMDYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR (SEQ ID NO: 16)
CD8 signal sequence (SEQ ID NO: 8 (amino acids 1-21 of SEQ ID NO: 16));
MALPVTALLLPLALLLHAARP (SEQ ID NO: 88)
Anti-CD37 H-L (SEQ ID NO: 4 (amino acids 22-265 of SEQ ID NO: 16));
AVQLVQSGAEVKKPGSSVKVSCKASGYSFTGYNMNWVRQAPGQGLEWMGNIDPYYGGTTYN
RKFKG RVTLTVDKSSSTAYMELSSLRSEDTAVYYCARSVGPMDYWGQGTLVTVSSGGGGSGG
GGSGGGGSGGGGSDIQMTQSPSSLSASVGDRVTITCRTSENVYSYLAWYQQKPGKAPKLLVSS
AKTLAEGVPSRFSGSGSGTDFTLTISSLQPEDFATYFCQHHSDNPWTFGQGTKVEIKR (SEQ ID NO: 4)
Linker region (SEQ ID NO: 3 (amino acids 266-285 of SEQ ID NO: 16));
GGGGSGGGGSGGGGSGGGGS (SEQ ID NO: 3)
Anti-CD19 (SEQ ID NO: 14 (amino acids 286-532 of SEQ ID NO: 16));
EIVMTQSPATLSLSPGERATLSCRASQDISKYLNWYQQKPGQAPRLLIYHTSRLHSGIPARFSGS
GSGTDYTLTISSLQPEDFAVYFCQQGNTLPYTFGQGTKLEIKGGGGSGGGGSGGGGSGGGGS
QVQLQESGPGLVKPSETLSLTCTVSGVSLPDYGVSWIRQPPGKGLEWIGVIWGSETTYYQSSLK
SRVTISKDNSKNQVSLKLSSVTAADTAVYYCAKHYYYGGSYAMDYWGQGTLVTVSS (SEQ ID NO: 14)
CD8 hinge and TM domains (SEQ ID NO: 9 (amino acids 533-601 of SEQ ID NO: 16));
TTTPAPRPPTPAPTIASQPLSLRPEACRPAAGGAVHTRGLDFACDIYIWAPLAGTCGVLLLSLVITL
YC (SEQ ID NO: 9)
4-1BB (SEQ ID NO: 10 (amino acids 602-643 of SEQ ID NO: 16)),
KRGRKKLLYIFKQPFMRPVQTTQEEDGCSCRFPEEEEGGCEL (SEQ ID NO: 10)
CD3ζ (SEQ ID NO: 11 (amino acids 644-755 of SEQ ID NO: 16)).
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR (SEQ ID NO: 11)

A table of the sequences of the application and the corresponding SEQ ID NO: is provided in Table 4.

TABLE 4
Sequences of the application

| SEQ ID | | |
|---|---|---|
| NO: | Sequence | Description |
| 1 | AVQLVQSGAEVKKPGSSVKVSCKASGYSFTGYNMNWVRQAPGQGLEWMGNIDP | Anti-CD37 VH |
| | YYGGTTYNRKFKGRVTLTVDKSSSTAYMELSSLRSEDTAVYYCARSVGPMDYWG | |
| | QGTLVTVSS | |
| 2 | DIQMTQSPSSLSASVGDRVTITCRTSENVYSYLAWYQQKPGKAPKLLVSSAKTLAE | Anti-CD37 VL |
| | GVPSRFSGSGSGTDFTLTISSLQPEDFATYFCQHHSDNPWTFGQGTKVEIKR | |
| 3 | GGGGSGGGGSGGGGSGGGGS | Linker |
| 4 | AVQLVQSGAEVKKPGSSVKVSCKASGYSFTGYNMNWVRQAPGQGLEWMGNIDP | Anti-CD37 scFv |
| | YYGGTTYNRKFKGRVTLTVDKSSSTAYMELSSLRSEDTAVYYCARSVGPMDYWG | VH-VL |
| | QGTLVTVSSGGGGSGGGGSGGGGSGGGGSDIQMTQSPSSLSASVGDRVTITCR | |
| | TSENVYSYLAWYQQKPGKAPKLLVSSAKTLAEGVPSRFSGSGSGTDFTLTISSLQP | |
| | EDFATYFCQHHSDNPWTFGQGTKVEIKR | |
| 5 | DIQMTQSPSSLSASVGDRVTITCRTSENVYSYLAWYQQKPGKAPKLLVSSAKTLAE | Anti-CD37 scFv |
| | GVPSRFSGSGSGTDFTLTISSLQPEDFATYFCQHHSDNPWTFGQGTKVEIKRGGG | VL-VH |
| | GSGGGGSGGGGSGGGGSAVQLVQSGAEVKKPGSSVKVSCKASGYSFTGYNMN | |
| | WVRQAPGQGLEWMGNIDPYYGGTTYNRKFKGRVTLTVDKSSSTAYMELSSLRSE | |
| | DTAVYYCARSVGPMDYWGQGTLVTVSS | |
| 6 | MALPVTALLLPLALLLHAARPAVQLVQSGAEVKKPGSSVKVSCKASGYSFTGYNM | CAR-37 H-L |
| | NWVRQAPGQGLEWMGNIDPYYGGTTYNRKFKGRVTLTVDKSSSTAYMELSSLRS | |
| | EDTAVYYCARSVGPMDYWGQGTLVTVSSGGGGSGGGGSGGGGSGGGGSDIQM | |
| | TQSPSSLSASVGDRVTITCRTSENVYSYLAWYQQKPGKAPKLLVSSAKTLAEGVP | |
| | SRFSGSGSGTDFTLTISSLQPEDFATYFCQHHSDNPWTFGQGTKVEIKRTTTPAPR | |
| | PPTPAPTIASQPLSLRPEACRPAAGGAVHTRGLDFACDIYIWAPLAGTCGVLLLSLV | |
| | ITLYCKRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQEGLYNE | |
| | LQKDKMAEAYSEIGMKGERRRGKGHDGLYQGLSTATKDTYDALHMQALPPR | |
| 7 | MALPVTALLLPLALLLHAARPDIQMTQSPSSLSASVGDRVTITCRTSENVYSYLAWY | CAR-37 L-H |
| | QQKPGKAPKLLVSSAKTLAEGVPSRFSGSGSGTDFTLTISSLQPEDFATYFCQHHS | |
| | DNPWTFGQGTKVEIKRGGGGSGGGGSGGGGSGGGGSAVQLVQSGAEVKKPGS | |
| | SVKVSCKASGYSFTGYNMNWVRQAPGQGLEWMGNIDPYYGGTTYNRKFKGRVT | |
| | LTVDKSSSTAYMELSSLRSEDTAVYYCARSVGPMDYWGQGTLVTVSSTTTPAPRP | |
| | PTPAPTIASQPLSLRPEACRPAAGGAVHTRGLDFACDIYIWAPLAGTCGVLLLSLVI | |
| | TLYCKRGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| 8 | MALPVTALLLPLALLLHAARP | CD8 leader |
| 9 | TTTPAPRPPTPAPTIASQPLSLRPEACRPAAGGAVHTRGLDFACDIYIWAPLAGTC | CD8 hinge/TM |
| | GVLLLSLVITLYC | |
| 10 | KRGRKKLLYIFKQPFMRPVQTTQEEDGCSCRFPEEEEGGCEL | 4-1BB |
| 11 | RVKFSRSADAPAYQQGQNQLYNELNLGRREEYDVLDKRRGRDPEMGGKPRRKN | CD3ζ |
| | PQEGLYNELQKDKMAEAYSEIGMKGERRRGKGHDGLYQGLSTATKDTYDALHMQ | |
| | ALPPR | |
| 12 | QVQLQESGPGLVKPSETLSLTCTVSGVSLPDYGVSWIRQPPGKGLEWIGVIWGSE | Anti-CD19 VH |
| | TTYYQSSLKSRVTISKDNSKNQVSLKLSSVTAADTAVYYCAKHYYYGGSYAMDYW | |
| | GQGTLVTVSS | |
| 13 | EIVMTQSPATLSLSPGERATLSCRASQDISKYLNWYQQKPGQAPRLLIYHTSRLHS | Anti-CD19 VL |
| | GIPARFSGSGSGTDYTLTISSLQPEDFAVYFCQQGNTLPYTFGQGTKLEIK | |
| 14 | EIVMTQSPATLSLSPGERATLSCRASQDISKYLNWYQQKPGQAPRLLIYHTSRLHS | Anti-CD19 scFv |
| | GIPARFSGSGSGTDYTLTISSLQPEDFAVYFCQQGNTLPYTFGQGTKLEIKGGGGS | |
| | GGGGSGGGGSGGGGSQVQLQESGPGLVKPSETLSLTCTVSGVSLPDYGVSWIR | |
| | QPPGKGLEWIGVIWGSETTYYQSSLKSRVTISKDNSKNQVSLKLSSVTAADTAVYY | |
| | CAKHYYYGGSYAMDYWGQGTLVTVSS | |
| 15 | MALPVTALLLPLALLLHAARPEIVMTQSPATLSLSPGERATLSCRASQDISKYLNWY | CAR-19-37 |
| | QQKPGQAPRLLIYHTSRLHSGIPARFSGSGSGTDYTLTISSLQPEDFAVYFCQQGN | |
| | TLPYTFGQGTKLEIKGGGGSGGGGSGGGGSGGGGSQVQLQESGPGLVKPSETL | |
| | SLTCTVSGVSLPDYGVSWIRQPPGKGLEWIGVIWGSETTYYQSSLKSRVTISKDNS | |
| | KNQVSLKLSSVTAADTAVYYCAKHYYYGGSYAMDYWGQGTLVTVSSGGGGSGG | |
| | GGSGGGGSGGGGSAVQLVQSGAEVKKPGSSVKVSCKASGYSFTGYNMNWVRQ | |
| | APGQGLEWMGNIDPYYGGTTYNRKFKGRVTLTVDKSSSTAYMELSSLRSEDTAVY | |
| | YCARSVGPMDYWGQGTLVTVSSGGGGSGGGGSGGGGSGGGGSDIQMTQSPSS | |
| | LSASVGDRVTITCRTSENVYSYLAWYQQKPGKAPKLLVSSAKTLAEGVPSRFSGS | |
| | GSGTDFTLTISSLQPEDFATYFCQHHSDNPWTFGQGTKVEIKRTTTPAPRPPTPAP | |
| | TIASQPLSLRPEACRPAAGGAVHTRGLDFACDIYIWAPLAGTCGVLLLSLVITLYCK | |
| | RGRKKLLYIFKQPFMRPVQTTQEEDGCSCRFPEEEEGGCELRVKFSRSADAPAYQ | |
| | QGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 16 | MALPVTALLLPLALLLHAARPAVQLVQSGAEVKKPGSSVKVSCKASGYSFTGYNM | CAR-37-19 |
| | NWVRQAPGQGLEWMGNIDPYYGGTTYNRKFKGRVTLTVDKSSSTAYMELSSLRS | |
| | EDTAVYYCARSVGPMDYWGQGTLVTVSSGGGGSGGGGSGGGGSGGGGSDIQM | |
| | TQSPSSLSASVGDRVTITCRTSENVYSYLAWYQQKPGKAPKLLVSSAKTLAEGVP | |
| | SRFSGSGSGTDFTLTISSLQPEDFATYFCQHHSDNPWTFGQGTKVEIKRGGGGSG | |
| | GGGSGGGGSGGGGSEIVMTQSPATLSLSPGERATLSCRASQDISKYLNWYQQKP | |
| | GQAPRLLIYHTSRLHSGIPARFSGSGSGTDYTLTISSLQPEDFAVYFCQQGNTLPYT | |
| | FGQGTKLEIKGGGGSGGGGSGGGGSGGGGSQVQLQESGPGLVKPSETLSLTCT | |
| | VSGVSLPDYGVSWIRQPPGKGLEWIGVIWGSETTYYQSSLKSRVTISKDNSKNQV | |
| | SLKLSSVTAADTAVYYCAKHYYYGGSYAMDYWGQGTLVTVSSTTTPAPRPPTPAP | |
| | TIASQPLSLRPEACRPAAGGAVHTRGLDFACDIYIWAPLAGTCGVLLLSLVITLYCK | |
| | RGRKKLLYIFKQPFMRPVQTTQEEDGCSCRFPEEEEGGCELRVKFSRSADAPAYQ | |
| | QGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 17 | AVQLVQSGAEVKKPGSSVKVSCKASGYSFTGYNMNWVRQAPGQGLEWMGNIDP | CAR-37H-L |
| | YYGGTTYNRKFKGRVTLTVDKSSSTAYMELSSLRSEDTAVYYCARSVGPMDYWG | (without CD8 |
| | QGTLVTVSSGGGGSGGGGSGGGGSGGGGSDIQMTQSPSSLSASVGDRVTITCR | leader) |
| | TSENVYSYLAWYQQKPGKAPKLLVSSAKTLAEGVPSRFSGSGSGTDFTLTISSLQP | |
| | EDFATYFCQHHSDNPWTFGQGTKVEIKRTTTPAPRPPTPAPTIASQPLSLRPEACR | |
| | PAAGGAVHTRGLDFACDIYIWAPLAGTCGVLLLSLVITLYCKRGRKKLLYIFKQPFM | |
| | RPVQTTQEEDGCSCRFPEEEEGGCELRVKFSRSADAPAYQQGQNQLYNELNLGR | |
| | REEYDVLDKRRGRDPEMGGKPRRKNPQEGLYNELQKDKMAEAYSEIGMKGERR | |
| | RGKGHDGLYQGLSTATKDTYDALHMQALPPR | |
| 18 | DIQMTQSPSSLSASVGDRVTITCRTSENVYSYLAWYQQKPGKAPKLLVSSAKTLAE | CAR-37 L-H |
| | GVPSRFSGSGSGTDFTLTISSLQPEDFATYFCQHHSDNPWTFGQGTKVEIKRGGG | (without CD8 |
| | GSGGGGSGGGGSGGGGSAVQLVQSGAEVKKPGSSVKVSCKASGYSFTGYNMN | leader) |
| | WVRQAPGQGLEWMGNIDPYYGGTTYNRKFKGRVTLTVDKSSSTAYMELSSLRSE | |
| | DTAVYYCARSVGPMDYWGQGTLVTVSSTTTPAPRPPTPAPTIASQPLSLRPEACR | |
| | PAAGGAVHTRGLDFACDIYIWAPLAGTCGVLLLSLVITLYCKRGRKKLLYIFKQPFM | |
| | RPVQTTQEEDGCSCRFPEEEEGGCELRVKFSRSADAPAYQQGQNQLYNELNLGR | |
| | REEYDVLDKRRGRDPEMGGKPRRKNPQEGLYNELQKDKMAEAYSEIGMKGERR | |
| | RGKGHDGLYQGLSTATKDTYDALHMQALPPR | |
| 19 | EIVMTQSPATLSLSPGERATLSCRASQDISKYLNWYQQKPGQAPRLLIYHTSRLHS | CAR-19-37 |
| | GIPARFSGSGSGTDYTLTISSLQPEDFAVYFCQQGNTLPYTFGQGTKLEIKGGGGS | (without CD8 |
| | GGGGSGGGGSGGGGSQVQLQESGPGLVKPSETLSLTCTVSGVSLPDYGVSWIR | leader) |
| | QPPGKGLEWIGVIWGSETTYYQSSLKSRVTISKDNSKNQVSLKLSSVTAADTAVYY | |
| | CAKHYYYGGSYAMDYWGQGTLVTVSSGGGGSGGGGSGGGGSGGGGSAVQLV | |
| | QSGAEVKKPGSSVKVSCKASGYSFTGYNMNWVRQAPGQGLEWMGNIDPYYGGT | |
| | TYNRKFKGRVTLTVDKSSSTAYMELSSLRSEDTAVYYCARSVGPMDYWGQGTLV | |
| | TVSSGGGGSGGGGSGGGGSGGGGSDIQMTQSPSSLSASVGDRVTITCRTSENV | |
| | YSYLAWYQQKPGKAPKLLVSSAKTLAEGVPSRFSGSGSGTDFTLTISSLQPEDFAT | |
| | YFCQHHSDNPWTFGQGTKVEIKRTTTPAPRPPTPAPTIASQPLSLRPEACRPAAG | |
| | GAVHTRGLDFACDIYIWAPLAGTCGVLLLSLVITLYCKRGRKKLLYIFKQPFMRPVQ | |
| | TTQEEDGCSCRFPEEEEGGCELRVKFSRSADAPAYQQGQNQLYNELNLGRREEY | |
| | DVLDKRRGRDPEMGGKPRRKNPQEGLYNELQKDKMAEAYSEIGMKGERRRGKG | |
| | HDGLYQGLSTATKDTYDALHMQALPPR | |
| 20 | AVQLVQSGAEVKKPGSSVKVSCKASGYSFTGYNMNWVRQAPGQGLEWMGNIDP | CAR-37-19 |
| | YYGGTTYNRKFKGRVTLTVDKSSSTAYMELSSLRSEDTAVYYCARSVGPMDYWG | (without CD8 |
| | QGTLVTVSSGGGGSGGGGSGGGGSGGGGSDIQMTQSPSSLSASVGDRVTITCR | leader) |
| | TSENVYSYLAWYQQKPGKAPKLLVSSAKTLAEGVPSRFSGSGSGTDFTLTISSLQP | |
| | EDFATYFCQHHSDNPWTFGQGTKVEIKRGGGGSGGGGSGGGGSGGGGSEIVMT | |
| | QSPATLSLSPGERATLSCRASQDISKYLNWYQQKPGQAPRLLIYHTSRLHSGIPAR | |
| | FSGSGSGTDYTLTISSLQPEDFAVYFCQQGNTLPYTFGQGTKLEIKGGGGSGGGG | |
| | SGGGGSGGGGSQVQLQESGPGLVKPSETLSLTCTVSGVSLPDYGVSWIRQPPGK | |
| | GLEWIGVIWGSETTYYQSSLKSRVTISKDNSKNQVSLKLSSVTAADTAVYYCAKHY | |
| | YYGGSYAMDYWGQGTLVTVSSTTTPAPRPPTPAPTIASQPLSLRPEACRPAAGGA | |
| | VHTRGLDFACDIYIWAPLAGTCGVLLLSLVITLYCKRGRKKLLYIFKQPFMRPVQTT | |
| | QEEDGCSCRFPEEEEGGCELRVKFSRSADAPAYQQGQNQLYNELNLGRREEYD | |
| | VLDKRRGRDPEMGGKPRRKNPQEGLYNELQKDKMAEAYSEIGMKGERRRGKGH | |
| | DGLYQGLSTATKDTYDALHMQALPPR | |

Some embodiments of the technology described herein can be defined according to any of the following numbered paragraphs:
1. A chimeric antigen receptor (CAR) comprising (i) an extracellular domain comprising a CD37-binding domain and a CD19-binding domain, (ii) a transmembrane domain, and (iii) an intracellular signaling domain.
2. The CAR of paragraph 1, wherein the CD37-binding domain and/or the CD19-binding domain comprises an antibody, or an antigen binding fragment thereof.
3. The CAR of paragraph 1 or 2, wherein the CD37-binding domain and/or the CD19-binding domain comprises a single chain variable fragment (scFv).
4. The CAR of any one of paragraphs 1-3, wherein the CD19-binding domain is positioned N-terminal to the CD37-binding domain.
5. The CAR of any one of paragraphs 1-3, wherein the CD37-binding domain is positioned N-terminal to the CD19-binding domain.
6. The CAR of any one of paragraphs 1-5, wherein the CAR further comprises (iv) one or more co-stimulatory domains.
7. The CAR of any one of paragraphs 1-6, wherein the transmembrane domain comprises a hinge/transmembrane domain.
8. The CAR of paragraph 7, wherein the hinge/transmembrane domain comprises the hinge/transmembrane domain of CD8 or 4-1 BB.
9. The CAR of paragraph 8, wherein the hinge/transmembrane domain comprises the hinge/transmembrane domain of CD8, optionally comprising the amino acid sequence of SEQ ID NO: 9.
10. The CAR of any one of paragraphs 1-9, wherein the intracellular signaling domain comprises the intracellular signaling domain of TCRζ, FcRγ, FcRβ, CD3γ, CD3θ, CD3ε, CD3ζ, CD22, CD79a, CD79b, or CD66d.
11. The CAR of paragraph 10, wherein the intracellular signaling domain comprises the intracellular signaling domain of CD3ζ, optionally comprising the amino acid sequence of SEQ ID NO: 11.
12. The CAR of any one of paragraphs 6-11, wherein the co-stimulatory domain comprises the co-stimulatory domain of 4-1 BB, CD28, or OX-40.
13. The CAR of paragraph 12, wherein the co-stimulatory domain comprises the co-stimulatory domain of 4-1 BB, optionally comprising the amino acid sequence of SEQ ID NO: 10.
14. The CAR of any one of paragraphs 1-13, wherein the CAR comprises an amino acid sequence having at least 90% sequence identity to the amino acid sequence of SEQ ID NO: 15, 16, 19, or 20.
15. The CAR of paragraph 14, wherein the CAR comprises the amino acid sequence of SEQ ID NO: 15, 16, 19, or 20.
16. The CAR of any one of paragraphs 1-15, wherein the CD37-binding domain comprises a heavy chain variable domain (VH) comprising an amino acid sequence having at least 90% sequence identity to the amino acid sequence of SEQ ID NO: 1 and a light chain variable domain (VL) comprising an amino acid sequence having at least 90% sequence identity to the amino acid sequence of SEQ ID NO: 2.
17. The CAR of paragraph 16, wherein the VH comprises the amino acid sequence of SEQ ID NO: 1 and the VL comprises the amino acid sequence of SEQ ID NO: 2.
18. The CAR of paragraph 16 or 17, wherein the VH is positioned N-terminal to the VL.
19. The CAR of paragraph 16 or 17, wherein the VL is positioned N-terminal to the VH.
20. The CAR of any one of paragraphs 1-19, wherein the CD37-binding domain comprises an amino acid sequence having at least 90% sequence identity to the amino acid sequence of SEQ ID NO: 4 or 5.
21. The CAR of paragraph 20, wherein the CD37-binding domain comprises the amino acid sequence of SEQ ID NO: 4 or 5.
22. The CAR of any one of paragraphs 1-21, wherein the CD19-binding domain comprises a heavy chain variable domain (VH) comprising an amino acid sequence having at least 90% sequence identity to the amino acid sequence of SEQ ID NO: 12 and a light chain variable domain (VL) comprising an amino acid sequence having at least 90% sequence identity to the amino acid sequence of SEQ ID NO: 13.
23. The CAR of paragraph 22, wherein the VH comprises the amino acid sequence of SEQ ID NO: 12 and the VL comprises the amino acid sequence of SEQ ID NO: 13.
24. The CAR of any one of paragraphs 1-23, wherein the CD19-binding domain comprises an amino acid sequence having at least 90% sequence identity to the amino acid sequence of SEQ ID NO: 14.
25. The CAR of paragraph 24, wherein the CD19-binding domain comprises the amino acid sequence of SEQ ID NO: 14.
26. A polynucleotide encoding the CAR of any one of paragraphs 1-25.
27. The polynucleotide of paragraph 26, further comprising a suicide gene.
28. The polynucleotide of paragraph 26 or 27, further comprising a sequence encoding a signal sequence.
29. An immune cell comprising the CAR of any one of paragraphs 1-25 and/or the polynucleotide of any one of paragraphs 26-28.
30. The immune cell of paragraph 29, wherein the immune cell is a T cell or a natural killer (NK) cell.
31. The immune cell of paragraph 29 or 30, wherein the immune cell is a human cell.
32. A pharmaceutical composition comprising the immune cell of any one of paragraphs 29-31 and a pharmaceutically acceptable carrier.
33. A method of treating a cancer in a subject in need thereof, the method comprising administering the immune cell of any one of paragraphs 29-31 or the pharmaceutical composition of paragraph 32 to the subject.
34. The method of paragraph 33, wherein the cancer comprises cells expressing CD37.
35. The method of paragraph 34, wherein the cancer is a B cell non-Hodgkin lymphoma, a T cell lymphoma, or a leukemia.
36. The method of paragraph 35, wherein the B cell non-Hodgkin lymphoma is mantle cell lymphoma (MCL), diffuse large B cell lymphoma (DLBCL), follicular lymphoma (FL), or Burkitt's lymphoma.

37. The method of paragraph 35, wherein the T cell lymphoma is peripheral T cell lymphoma (PTCL), cutaneous T cell lymphoma (CTCL), angioimmunoblastic T cell lymphoma (AITL), or anaplastic large cell lymphoma (ALCL).
38. The method of paragraph 35, wherein the leukemia is chronic lymphocytic leukemia (CLL).
39. The method of any one of paragraphs 33-38, wherein the subject is non-responsive to anti-CD19 therapy.
40. The method of any one of paragraphs 33-39, wherein the subject is co-administered anti-CD19 therapy.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A chimeric antigen receptor (CAR) comprising (i) an extracellular domain comprising a CD37-binding domain and a CD19-binding domain, (ii) a transmembrane domain, and (iii) an intracellular signaling domain.

2. The CAR of claim 1, wherein the CD37-binding domain and/or the CD19-binding domain comprises an antibody, or an antigen binding fragment thereof.

3. The CAR of claim 2, wherein the CD37-binding domain and/or the CD19-binding domain comprises a single chain variable fragment (scFv).

4. The CAR of claim 1, wherein the CD19-binding domain is positioned N-terminal to the CD37-binding domain.

5. The CAR of claim 1, wherein the CD37-binding domain is positioned N-terminal to the CD19-binding domain.

6. The CAR of claim 1, wherein the CAR further comprises (iv) one or more co-stimulatory domains.

7. The CAR of claim 1, wherein the transmembrane domain comprises a hinge/transmembrane domain.

8. The CAR of claim 7, wherein the hinge/transmembrane domain comprises the hinge/transmembrane domain of CD8 or 4-1 BB.

9. The CAR of claim 8, wherein the hinge/transmembrane domain comprises the hinge/transmembrane domain of CD8.

10. The CAR of claim 1, wherein the intracellular signaling domain comprises the intracellular signaling domain of TCRζ, FcRγ, FcRβ, CD3γ, CD3θ, CD3ε, CD3ζ, CD22, CD79a, CD79b, or CD66d.

11. The CAR of claim 10, wherein the intracellular signaling domain comprises the intracellular signaling domain of CD3ζ.

12. The CAR of claim 6, wherein the co-stimulatory domain comprises the co-stimulatory domain of 4-1 BB, CD28, or OX-40.

13. The CAR of claim 12, wherein the co-stimulatory domain comprises the co-stimulatory domain of 4-1 BB.

14. The CAR of claim 1, wherein the CAR comprises an amino acid sequence having at least 90% sequence identity to the amino acid sequence of SEQ ID NO: 20.

15. The CAR of claim 14, wherein the CAR comprises the amino acid sequence of SEQ ID NO: 20.

16. The CAR of claim 1, wherein the CD37-binding domain comprises a heavy chain variable domain (VH) comprising an amino acid sequence having at least 90% sequence identity to the amino acid sequence of SEQ ID NO: 1 and a light chain variable domain (VL) comprising an amino acid sequence having at least 90% sequence identity to the amino acid sequence of SEQ ID NO: 2.

17. The CAR of claim 16, wherein the VH comprises the amino acid sequence of SEQ ID NO: 1 and the VL comprises the amino acid sequence of SEQ ID NO: 2.

18. The CAR of claim 16, wherein the VH is positioned N-terminal to the VL.

19. The CAR of claim 16, wherein the VL is positioned N-terminal to the VH.

20. The CAR of claim 1, wherein the CD37-binding domain comprises an amino acid sequence having at least 90% sequence identity to the amino acid sequence of SEQ ID NO: 4 or 5.

21. The CAR of claim 20, wherein the CD37-binding domain comprises the amino acid sequence of SEQ ID NO: 4 or 5.

22. The CAR of claim 1, wherein the CD19-binding domain comprises a heavy chain variable domain (VH) comprising an amino acid sequence having at least 90% sequence identity to the amino acid sequence of SEQ ID NO: 12 and a light chain variable domain (VL) comprising an amino acid sequence having at least 90% sequence identity to the amino acid sequence of SEQ ID NO: 13.

23. The CAR of claim 22, wherein the VH comprises the amino acid sequence of SEQ ID NO: 12 and the VL comprises the amino acid sequence of SEQ ID NO: 13.

24. The CAR of claim 1, wherein the CD19-binding domain comprises an amino acid sequence having at least 90% sequence identity to the amino acid sequence of SEQ ID NO: 14.

25. The CAR of claim 24, wherein the CD19-binding domain comprises the amino acid sequence of SEQ ID NO: 14.

26. A polynucleotide encoding the CAR of claim 1.

27. The polynucleotide of claim 26, further comprising a suicide gene.

28. The polynucleotide of claim 26, further comprising a sequence encoding a signal sequence.

29. An immune cell comprising the CAR of claim 1 and/or a polynucleotide encoding the CAR of claim 1.

30. The immune cell of claim 29, wherein the immune cell is a T cell or a natural killer (NK) cell.

31. The immune cell of claim 29, wherein the immune cell is a human cell.

32. A pharmaceutical composition comprising the immune cell of claim 29 and a pharmaceutically acceptable carrier.

33. A method of treating a cancer in a subject in need thereof, the method comprising administering the immune cell of claim 29, or a pharmaceutical composition thereof, to the subject.

34. The method of claim 33, wherein the cancer comprises cells expressing CD37.

35. The method of claim 34, wherein the cancer is a B cell non-Hodgkin lymphoma, a T cell lymphoma, or a leukemia.

36. The method of claim 35, wherein the B cell non-Hodgkin lymphoma is mantle cell lymphoma (MCL), diffuse large B cell lymphoma (DLBCL), follicular lymphoma (FL), or Burkitt's lymphoma.

37. The method of claim 35, wherein the T cell lymphoma is peripheral T cell lymphoma (PTCL), cutaneous T cell lymphoma (CTCL), angioimmunoblastic T cell lymphoma (AITL), or anaplastic large cell lymphoma (ALCL).

38. The method of claim 35, wherein the leukemia is chronic lymphocytic leukemia (CLL).

39. The method of claim 33, wherein the subject is non-responsive to anti-CD19 therapy.

40. The method of claim 33, wherein the subject is co-administered anti-CD19 therapy.
